(19) United States
(12) Patent Application Publication
(10) Pub. No.: US 2022/0304972 A1
(43) Pub. Date: Sep. 29, 2022

(54) COMBINATION THERAPY AND BIOMARKER INDICATING EFFICACY THEREOF

(71) Applicants: Sumitomo Dainippon Pharma Co., Ltd., Osaka-shi (JP); National Cancer Center, Chuo-ku (JP)

(72) Inventors: Takayuki YOSHINO, Kashiwa-shi (JP); Yasutoshi KUBOKI, Kashiwa-shi (JP); Akihito KAWAZOE, Kashiwa-shi (JP)

(73) Assignees: Sumitomo Dainippon Pharma Co., Ltd., Osaka-shi (JP); National Cancer Center, Chuo-ku (JP)

(21) Appl. No.: 17/637,014

(22) PCT Filed: Aug. 21, 2020
(86) PCT No.: PCT/JP2020/031572

§ 371 Date: Feb. 21, 2022

(30) Foreign Application Priority Data
Aug. 23, 2019 (JP) 2019-153109

(51) Int. Cl. A61K 31/343; A61K 45/06; G01N 33/574; A61P 35/00; A61K 31/519

(52) U.S. Cl. CPC A61K 31/343; A61K 45/06; G01N 33/574; A61P 35/00; A61K 31/519

(57) ABSTRACT

The present disclosure provides a method for treating a microsatellite stable cancer patient with a specific combination of medical agents or a composition or combination therefor. Specific combinations of medical agents include a combination of a cancer stem cell inhibitor (e.g., napabucasin) and an immune checkpoint inhibitor (e.g., pembrolizumab). The MSS patient can be selected by determining if the patient has one or more patient characteristics. Another aspect of the disclosure provides a method for predicting responsiveness of a patient to a cancer treatment based on one or more patient characteristics.

### TECHNICAL FIELD

The present disclosure relates to the field of cancer therapy.

### BACKGROUND

Immune checkpoint inhibitors are attracting much attention in cancer therapy. Pembrolizumab, a leading anti-PD-1 antibody, is approved by the U.S. Food and Drug Administration (FDA) for the treatment of solid tumors with high frequency microsatellite instability (MSI-H) or mismatch repair deficiency (dMMR). However, pembrolizumab has not shown promising antitumor effects in microsatellite stable (MSS) metastatic colorectal cancer patients, and there is still a high unmet medical need.

### SUMMARY OF INVENTION

### Means to Solve Problem

The present disclosure provides a method for treating a MSS cancer patient with a specific combination of medical agents, or a composition or combination therefor. Specific combinations of medical agents include a combination of a cancer stem cell inhibitor (e.g., napabucasin) and an immune checkpoint inhibitor (e.g., pembrolizumab). The MSS patient can be selected by determining if the patient has one or more patient characteristics. The patient characteristics include characteristics that the cancer is right-sided colorectal cancer, that PD-L1 expression is positive on tumor cells, that PD-L1 expression is positive on immune cells, and that Consensus Molecular Subtypes (CMS) is 1 or 4. Another aspect of the disclosure provides a method for predicting responsiveness of a patient to a cancer treatment based on one or more patient characteristics.

Examples of embodiments of the present disclosure are shown in the following items.

1. A combination for treating a microsatellite stable (MSS) cancer patient, the combination comprising a cancer stem cell inhibitor and an immune checkpoint inhibitor, wherein the combination is administered to a patient having one or more patient characteristics that indicate that the patient is responsive to the treatment.

2. A composition comprising a cancer stem cell inhibitor for treating a microsatellite stable (MSS) cancer patient, wherein the composition is administered in combination with an immune checkpoint inhibitor to a patient having one or more patient characteristics that indicate that the patient is responsive to the treatment.

3. A composition comprising an immune checkpoint inhibitor for treating a microsatellite stable (MSS) cancer patient, wherein the composition is administered in combination with a cancer stem cell inhibitor to a patient having one or more patient characteristics that indicate that the patient is responsive to the treatment.

4. The combination or composition according to any one of Items 1 to 3, wherein the one or more patient characteristics comprise a characteristic that the cancer is colorectal cancer.

5. The combination or composition according to Item 4, wherein the one or more patient characteristics comprise a characteristic that the cancer is right-sided colorectal cancer.

6. The combination or composition according to any one of Items 1 to 5, wherein the one or more patient characteristics comprise a characteristic that PD-L1 expression is positive on immune cells of the patient.

7. The combination or composition according to any one of Items 1 to 6, wherein the one or more patient characteristics comprise a characteristic that PD-L1 expression is positive on tumor cells of the patient.

8. The combination or composition according to any one of Items 1 to 7, wherein the one or more patient characteristics comprise a characteristic that CMS of the patient is 1 or 4.

9. The combination or composition according to any one of Items 1 to 3, wherein the one or more patient characteristics comprise characteristics
(1) the cancer is right-sided colorectal cancer, and PD-L1 expression is positive on immune cells of the patient,
(2) the cancer is right-sided colorectal cancer, and PD-L1 expression is positive on tumor cells of the patient, or
(3) the cancer is right-sided colorectal cancer, and CMS of the patient is 1 or 4.

10. The combination or composition according to any one of Items 1 to 9, wherein the immune checkpoint inhibitor is an inhibitor of PD-L1 or PD-1.

11. The combination or composition according to any one of Items 1 to 10, wherein the immune checkpoint inhibitor is an anti-PD-1 antibody.

12. The combination or composition according to any one of Items 1 to 11, wherein the cancer stem cell inhibitor is a STAT3 pathway inhibitor.

13. The combination or composition according to any one of Items 1 to 12, wherein the cancer stem cell inhibitor is

a compound of Formula I:

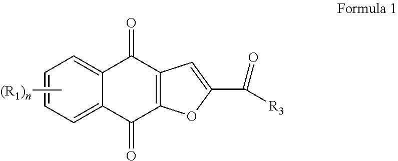

wherein,

each (R₁) is independently selected from the group consisting of hydrogen, halogen, fluorine, cyano, nitro, CF₃, OCF₃, alkyl, methyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NH₂,

n is 0, 1, 2, 3, or 4,

R₃ is selected from the group consisting of hydrogen, halogen, fluorine, cyano, CF₃, OCF₃, alkyl, methyl, substituted alkyl, halogen-substituted alkyl, hydroxyl-substituted alkyl, amine-substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NR_{b}R_{c}, wherein Rₐ is independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, and substituted aryl, and R_{b} and R_{c} are independently selected from the group consisting of hydrogen, alkyl, substituted alkyl, cycloalkyl, substituted cycloalkyl, heterocycle, substituted heterocycle, aryl, and substituted aryl, or R_{b} and R_{c} form a heterocycle or a substituted heterocycle together with the N to which they are attached, or

a compound of Formula 1A:

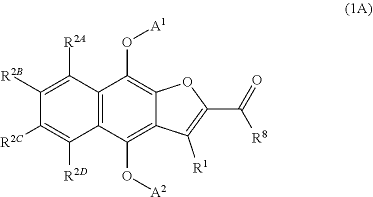

wherein A¹ and A² are the same or different and independently —C(═O)B, —CO₂B, —CONR^{3C}B, or a hydrogen atom, wherein A¹ and A² are not hydrogen atoms at the same time,

wherein B is

(1) a 3- to 6-membered monocyclic or polycyclic heterocyclic group,

(2) a 3- to 6-membered cyclic amino group, or

(3) a group represented by Formula (B):

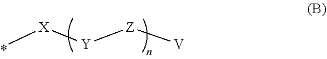

wherein the 3- to 6-membered monocyclic heterocyclic group and the 3- to 6-membered cyclic amino group have at least one secondary nitrogen atom in the ring, and

in Formula (B),

X is

(1) a single bond,

(2) C₁₋₆ alkylene, wherein the alkylene is optionally substituted with 1 to 3 substituents selected from the group consisting of a carboxyl group and —CO₂R⁶, or

(3) C₃₋₁₀ cycloalkylene,

Y is a single bond, an oxygen atom, or —NR^{4A}—,

R⁴A is a hydrogen atom,

Z is

(1) a single bond or

(2) C₁₋₆ alkylene,

n is 0 or 1,

V is

(1) —NHR⁵,

(2) a 3- to 6-membered monocyclic or polycyclic heterocyclic group, or

(3) a 3- to 6-membered cyclic amino group,

wherein the 3- to 6-membered monocyclic or polycyclic heterocyclic group and the 3- to 6-membered cyclic amino group have at least one secondary nitrogen atom in the ring,

R⁵ is

(1) a hydrogen atom or

(2) a C₁₋₆ alkyl group, wherein the alkyl group is optionally substituted with 1 to 3 substituents selected from the group consisting of a halogen atom, a hydroxyl group, a carboxyl group, a sulfinic acid group, a sulfonic acid group, a phosphoric acid group, a C₆₋₁₀ aryl group, a C₁₋₆ alkoxy group, a C₃₋₈ cycloalkoxy group, —NR⁶R⁷, —CO₂R⁶, —CONR⁶R⁷, —SO₂R⁶, —SO₂NR⁶R⁷, —OCO₂R⁶, —OCONR⁶R⁷ and —NR⁶CO₂R⁷, and

R⁶ and R⁷ are the same or different and independently a hydrogen atom or a C₁₋₆ alkyl group optionally substituted with 1 to 2 carboxyl groups, wherein, when both R⁶ and R⁷ are optionally substituted C₁₋₆ alkyl groups, they may form a 3- to 12-membered cyclic amino group together with the nitrogen atom to which they are attached, and

R^{3C} is a hydrogen atom;

R¹ is a hydrogen atom;

R^{2A}, R^{2B}, R^{2C} and R^{2D} are all hydrogen atoms;

R⁸ is a methyl group,

or a pharmaceutically acceptable salt or solvate thereof.

14. The combination or composition according to any one of Items 1 to 13, wherein the cancer stem cell inhibitor is napabucasin or a prodrug thereof, or a pharmaceutically acceptable salt thereof.

15. A combination for treating a microsatellite stable (MSS) cancer patient, the combination comprising napabucasin or a prodrug thereof, or a pharmaceutically acceptable salt thereof, and pembrolizumab, wherein the combination is administered to a patient having one or more patient characteristics that indicate that the patient is responsive to the treatment, and the one or more patient characteristics comprise characteristics
(1) the cancer is right-sided colorectal cancer, and PD-L1 expression is positive on immune cells of the patient,
(2) the cancer is right-sided colorectal cancer, and PD-L1 expression is positive on tumor cells of the patient, or
(3) the cancer is colorectal cancer, and CMS of the patient is 1 or 4.

16. A composition comprising napabucasin or a prodrug thereof, or a pharmaceutically acceptable salt thereof, for treating a microsatellite stable (MSS) cancer patient, wherein the composition is administered in combination with pembrolizumab to a patient having one or more patient characteristics that indicate that the patient is responsive to the treatment, and the one or more patient characteristics comprise characteristics
(1) the cancer is right-sided colorectal cancer, and PD-L1 expression is positive on immune cells of the patient,
(2) the cancer is right-sided colorectal cancer, and PD-L1 expression is positive on tumor cells of the patient, or
(3) the cancer is colorectal cancer, and CMS of the patient is 1 or 4.

17. A composition comprising pembrolizumab for treating a microsatellite stable (MSS) cancer patient, wherein the composition is administered in combination with napabucasin or a prodrug thereof, or a pharmaceutically acceptable salt thereof, to a patient having one or more patient characteristics that indicate that the patient is responsive to the treatment, and the one or more patient characteristics comprise characteristics
(1) the cancer is right-sided colorectal cancer, and PD-L1 expression is positive on immune cells of the patient,
(2) the cancer is right-sided colorectal cancer, and PD-L1 expression is positive on tumor cells of the patient, or
(3) the cancer is colorectal cancer, and CMS of the patient is 1 or 4.

18. A composition for determining responsiveness of a microsatellite stable (MSS) cancer patient to a cancer treatment comprising combination use of a cancer stem cell inhibitor and an immune checkpoint inhibitor, the composition comprising a detection agent for PD-L1, wherein a characteristic that PD-L1 expression is positive on tumor cells of the patient; or that PD-L1 expression is positive on immune cells of the patient indicates that the patient is responsive to the cancer treatment.

19. The composition according to Item 18, wherein the detection agent for PD-L1 is an anti-PD-L1 antibody.

20. A kit comprising the composition according to Item 18 or 19.

21. A method, comprising using one or more patient characteristics in a microsatellite stable (MSS) cancer patient as an indicator of responsiveness of the patient to a cancer treatment, wherein the cancer treatment comprises combination use of a cancer stem cell inhibitor and an immune checkpoint inhibitor and the presence of the one or more patient characteristics indicates that the patient is responsive to the cancer treatment.

22. The method according to Item 21, wherein the one or more patient characteristics comprise a characteristic that the cancer is right-sided colorectal cancer.

23. The method according to Item 21, wherein the one or more patient characteristics comprise a characteristic that PD-L1 expression is positive in the patient.

24. The method according to Item 21, wherein the one or more patient characteristics comprise a characteristic that PD-L1 expression is positive on tumor cells of the patient.

25. The method according to Item 21, wherein the one or more patient characteristics comprise a characteristic that CMS of the patient is 1 or 4.

26. A combination for treating a microsatellite stable (MSS) cancer patient, the combination comprising a reactive oxygen species generator (ROS generator) and an immune checkpoint inhibitor, wherein the combination is administered to a patient having one or more patient characteristics that indicate that the patient is responsive to the treatment.

27. A composition comprising a reactive oxygen species generator (ROS generator) for treating a microsatellite stable (MSS) cancer patient, wherein the composition is administered in combination with an immune checkpoint inhibitor to a patient having one or more patient characteristics that indicate that the patient is responsive to the treatment.

28. A composition comprising an immune checkpoint inhibitor for treating a microsatellite stable (MSS) cancer patient, wherein the composition is administered in combination with a reactive oxygen species generator (ROS generator) to a patient having one or more patient characteristics that indicate that the patient is responsive to the treatment.

29. The combination or composition according to any one of Items 26 to 28, wherein the one or more patient characteristics comprise a characteristic that the cancer is right-sided colorectal cancer.

30. The combination or composition according to any one of Items 26 to 28, wherein the one or more patient characteristics comprise a characteristic that PD-L1 expression is positive on immune cells of the patient.

31. The combination or composition according to any one of Items 26 to 28, wherein the one or more patient characteristics comprise a characteristic that PD-L1 expression is positive on tumor cells of the patient.

32. The combination or composition according to any one of Items 26 to 28, wherein the one or more patient characteristics comprise a characteristic that CMS of the patient is 1 or 4.

33. The combination or composition according to any one of Items 26 to 32, wherein the one or more patient characteristics comprise a characteristic that the cancer is right-sided colorectal cancer, that PD-L1 expression is positive on tumor cells of the patient, that PD-L1 expression is positive on immune cells of the patient, or that CMS of the patient is 1 or 4.

34. The combination or composition according to any one of Items 26 to 32, wherein the one or more patient characteristics comprise a characteristic that the cancer is right-sided colorectal cancer, and a characteristic that PD-L1 expression is positive on tumor cells of the patient or that PD-L1 expression is positive on immune cells of the patient.

35. The combination or composition according to any one of Items 26 to 34, wherein the immune checkpoint inhibitor is an inhibitor of PD-L1 or PD-1.

36. The combination or composition according to any one of Items 26 to 35, wherein the immune checkpoint inhibitor is an anti-PD-1 antibody.

37. The combination or composition according to any one of Items 26 to 36, wherein the reactive oxygen species generator (ROS generator) has a STAT3 pathway inhibitory activity.

38. The combination or composition according to any one of Items 26 to 36, wherein the reactive oxygen species generator is

a compound of Formula I :

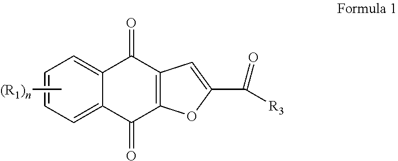

wherein,

each (R₁) is independently selected from the group consisting of hydrogen, halogen, fluorine, cyano, nitro, CF₂, OCF₃, alkyl, methyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NH₂,

n is 0, 1, 2, 3, or 4,

R₃ is selected from the group consisting of hydrogen, halogen, fluorine, cyano, CF₃, OCF₃, alkyl, methyl, substituted alkyl, halogen-substituted alkyl, hydroxyl-substituted alkyl, amine-substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NR_{b}R_{c}, wherein Rₐ is independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, and substituted aryl, and R_{b} and R_{c} are independently selected from the group consisting of hydrogen, alkyl, substituted alkyl, cycloalkyl, substituted cycloalkyl, heterocycle, substituted heterocycle, aryl, and substituted aryl, or R_{b} and R_{c} form a heterocycle or a substituted heterocycle together with the N to which they are attached, or

a compound of Formula 1A:

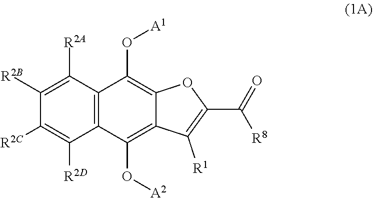

wherein A¹ and A² are the same or different and independently —C(═O)B, —CO₂B, —CONR^{3C}B, or a hydrogen atom, wherein A¹ and A² are not hydrogen atoms at the same time,

wherein B is

(1) a 3- to 6-membered monocyclic or polycyclic heterocyclic group,

(2) a 3- to 6-membered cyclic amino group, or

(3) a group represented by Formula (B):

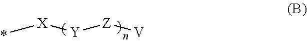

wherein the 3- to 6-membered monocyclic heterocyclic group and the 3- to 6-membered cyclic amino group have at least one secondary nitrogen atom in the ring, and

in Formula (B),

X is

(1) a single bond,

(2) C₁₋₆ alkylene, wherein the alkylene is optionally substituted with 1 to 3 substituents selected from the group consisting of a carboxyl group and —CO₂R⁶, or

(3) C₃₋₁₀ cycloalkylene,

Y is a single bond, an oxygen atom, or —NR^{4A}—,

R^{4A} is a hydrogen atom,

Z is

(1) a single bond or

(2) C₁₋₆ alkylene,

n is 0 or 1,

V is

(1) —NHR⁵,

(2) a 3- to 6-membered monocyclic or polycyclic heterocyclic group, or

(3) a 3- to 6-membered cyclic amino group,

wherein the 3- to 6-membered monocyclic or polycyclic heterocyclic group and the 3- to 6-membered cyclic amino group have at least one secondary nitrogen atom in the ring,

R⁵ is

(1) a hydrogen atom or

(2) a C₁₋₆ alkyl group, wherein the alkyl group is optionally substituted with 1 to 3 substituents selected from the group consisting of a halogen atom, a hydroxyl group, a carboxyl group, a sulfinic acid group, a sulfonic acid group, a phosphoric acid group, a C₆₋₁₀ aryl group, a C₁₋₆ alkoxy group, a C₃₋₈ cycloalkoxy group, —NR⁶R⁷, —CO₂R⁶, —CONR⁶R⁷, —SO₂R⁶, —SO₂NR⁶R⁷, —OCO₂R⁶, —OCONR⁶R⁷ and —NR⁶CO₂R⁷, and

R⁶ and R⁷ are the same or different and independently a hydrogen atom or a C₁₋₆ alkyl group optionally substituted with 1 to 2 carboxyl groups, wherein, when both R⁶ and R⁷ are optionally substituted C₁₋₆ alkyl groups, they may form a 3- to 12-membered cyclic amino group together with the nitrogen atom to which they are attached, and

R^{3C} is a hydrogen atom;

R¹ is a hydrogen atom;

R^{2A}, R^{2B}, R^{2C} and R^{2D} are all hydrogen atoms;

R⁸ is a methyl group,

or a pharmaceutically acceptable salt or solvate thereof.

39. The combination or composition according to any one of Items 26 to 38, wherein the reactive oxygen species generator (ROS generator) is napabucasin or a prodrug thereof, or a pharmaceutically acceptable salt thereof.

40. A composition for determining responsiveness of a microsatellite stable (MSS) cancer patient to a cancer treatment comprising combination use of a reactive oxygen species generator (ROS generator) and an immune checkpoint inhibitor, the composition comprising a detection agent for PD-L1, wherein a characteristic that PD-L1 expression is positive on tumor cells of the patient; or that PD-L1 expression is positive on immune cells of the patient indicates that the patient is responsive to the cancer treatment.

41. The composition according to Item 40, wherein the detection agent for PD-L1 is an anti-PD-L1 antibody.

42. A kit comprising the composition according to Item 40 or 41.

43. A method, comprising using one or more patient characteristics in a microsatellite stable (MSS) cancer patient as an indicator of responsiveness of the patient to a cancer treatment, wherein the cancer treatment comprises combination use of a reactive oxygen species generator (ROS generator) and an immune checkpoint inhibitor and the presence of the one or more patient characteristics indicates that the patient is responsive to the cancer treatment.

44. The method according to Item 43, wherein the one or more patient characteristics comprise a characteristic that the cancer is colorectal cancer.

45. The method according to Item 44, wherein the one or more patient characteristics comprise a characteristic that the cancer is right-sided colorectal cancer.

46. The method according to any one of Items 43 to 45, wherein the one or more patient characteristics comprise a characteristic that PD-L1 expression is positive on immune cells of the patient.

47. The method according to any one of Items 43 to 46, wherein the one or more patient characteristics comprise a characteristic that PD-L1 expression is positive on tumor cells of the patient.

48. The method according to any one of Items 43 to 47, wherein the one or more patient characteristics comprise a characteristic that CMS of the patient is 1 or 4.

49. A method for treating a microsatellite stable (MSS) cancer patient, comprising a step of administering to the patient an effective amount of a cancer stem cell inhibitor and an effective amount of an immune checkpoint inhibitor in combination, wherein the patient has one or more patient characteristics that indicate that the patient is responsive to the cancer treatment.

50. The method according to Item 49, wherein the cancer stem cell inhibitor is administered separately from the immune checkpoint inhibitor.

51. The method according to Item 49, wherein the cancer stem cell inhibitor is administered together with the immune checkpoint inhibitor.

52. The method according to any one of Items 49 to 51, wherein the one or more patient characteristics comprise a characteristic that the cancer is colorectal cancer.

53. The method according to Item 52, wherein the one or more patient characteristics comprise a characteristic that the cancer is right-sided colorectal cancer.

54. The method according to any one of Items 49 to 53, wherein the one or more patient characteristics comprise a characteristic that PD-L1 expression is positive on immune cells of the patient.

55. The method according to any one of Items 49 to 54, wherein the one or more patient characteristics comprise a characteristic that PD-L1 expression is positive on tumor cells of the patient.

56. The method according to any one of Items 49 to 55, wherein the one or more patient characteristics comprise a characteristic that CMS of the patient is 1 or 4.

57. The method according to any one of Items 49 to 51, wherein the one or more patient characteristics comprise characteristics
(1) the cancer is right-sided colorectal cancer, and PD-L1 expression is positive on immune cells of the patient,
(2) the cancer is right-sided colorectal cancer, and PD-L1 expression is positive on tumor cells of the patient, or
(3) the cancer is right-sided colorectal cancer, and CMS of the patient is 1 or 4.

58. The method according to any one of Items 49 to 57, wherein the immune checkpoint inhibitor is an inhibitor of PD-L1 or PD-1.

59. The method according to any one of Items 49 to 58, wherein the immune checkpoint inhibitor is an anti-PD-1 antibody.

60. The method according to any one of Items 49 to 59, wherein the cancer stem cell inhibitor is a STAT3 pathway inhibitor.

61. The method according to any one of Items 49 to 60, wherein the cancer stem cell inhibitor is

a compound of Formula I:

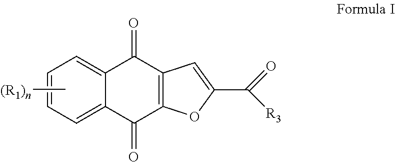

wherein,

each (R₁) is independently selected from the group consisting of hydrogen, halogen, fluorine, cyano, nitro, CF₃, OCF₃, alkyl, methyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NH₂,

n is 0, 1, 2, 3, or 4,

R₃ is selected from the group consisting of hydrogen, halogen, fluorine, cyano, CF₃, OCF₃, alkyl, methyl, substituted alkyl, halogen-substituted alkyl, hydroxyl-substituted alkyl, amine-substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NR_{b}R_{c}, wherein Rₐ is independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, and substituted aryl, and R_{b} and R_{c} are independently selected from the group consisting of hydrogen, alkyl, substituted alkyl, cycloalkyl, substituted cycloalkyl, heterocycle, substituted heterocycle, aryl, and substituted aryl, or R_{b} and R_{c} form a heterocycle or a substituted heterocycle together with the N to which they are attached, or

a compound of Formula 1A:

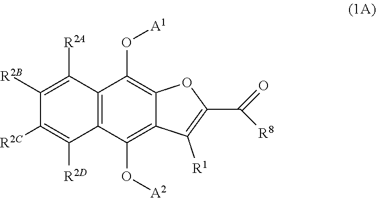

wherein A¹ and A² are the same or different and independently —C(═O)B, —CO₂B, —CONR^{3C}B, or a hydrogen atom, wherein A¹ and A² are not hydrogen atoms at the same time,

wherein B is

(1) a 3- to 6-membered monocyclic or polycyclic heterocyclic group,

(2) a 3- to 6-membered cyclic amino group, or

(3) a group represented by Formula (B):

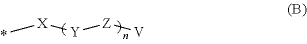

wherein the 3- to 6-membered monocyclic heterocyclic group and the 3- to 6-membered cyclic amino group have at least one secondary nitrogen atom in the ring, and

in Formula (B),

X is

(1) a single bond,

(2) C₁₋₆ alkylene, wherein the alkylene is optionally substituted with 1 to 3 substituents selected from the group consisting of a carboxyl group and —CO₂R⁶, or

(3) C₃₋₁₀ cycloalkylene,

Y is a single bond, an oxygen atom, or —NR^{4A}—,

R^{4A} is a hydrogen atom,

Z is

(1) a single bond or

(2) C₁₋₆ alkylene,

n is 0 or 1,

V is

(1) —NHR⁵,

(2) a 3- to 6-membered monocyclic or polycyclic heterocyclic group, or

(3) a 3- to 6-membered cyclic amino group,

wherein the 3- to 6-membered monocyclic or polycyclic heterocyclic group and the 3- to 6-membered cyclic amino group have at least one secondary nitrogen atom in the ring,

R⁵ is

(1) a hydrogen atom or

(2) a C₁₋₆ alkyl group, wherein the alkyl group is optionally substituted with 1 to 3 substituents selected from the group consisting of a halogen atom, a hydroxyl group, a carboxyl group, a sulfinic acid group, a sulfonic acid group, a phosphoric acid group, a C₈₋₁₀ aryl group, a C₁₋₆ alkoxy group, a C₃₋₈ cycloalkoxy group, —NR⁶R⁷, —CO₂R⁶, —CONR⁶R⁷, —SO₂R⁶, —SO₂NR⁶R⁷, —OCO₂R⁶, —OCONR⁶R⁷ and —NR⁶CO₂R⁷, and

R⁶ and R⁷ are the same or different and independently a hydrogen atom or a C₁₋₆ alkyl group optionally substituted with 1 to 2 carboxyl groups, wherein, when both R⁶ and R⁷ are optionally substituted C₁₋₆ alkyl groups, they may form a 3- to 12-membered cyclic amino group together with the nitrogen atom to which they are attached, and

R^{3C} is a hydrogen atom;

R¹ is a hydrogen atom;

R^{2A}, R^{2B}, R^{2C} and R^{2D} are all hydrogen atoms;

R⁸ is a methyl group,

or a pharmaceutically acceptable salt or solvate thereof.

62. The method according to any one of Items 49 to 61, wherein the cancer stem cell inhibitor is napabucasin or a prodrug thereof, or a pharmaceutically acceptable salt thereof.

63. A method for treating a microsatellite stable (MSS) cancer patient, comprising a step of administering to the patient a combination of napabucasin or a prodrug thereof, or a pharmaceutically acceptable salt thereof, and pembrolizumab, wherein the patient has one or more patient characteristics that indicate that the patient is responsive to the treatment, and the one or more patient characteristics comprise characteristics
(1) the cancer is right-sided colorectal cancer, and PD-L1 expression is positive on immune cells of the patient,
(2) the cancer is right-sided colorectal cancer, and PD-L1 expression is positive on tumor cells of the patient, or
(3) the cancer is colorectal cancer, and CMS of the patient is 1 or 4.

64. A method for treating a microsatellite stable (MSS) cancer patient, comprising a step of administering to the patient a composition comprising napabucasin or a prodrug thereof, or a pharmaceutically acceptable salt thereof, wherein the composition is administered in combination with pembrolizumab and the patient has one or more patient characteristics that indicate that the patient is responsive to the treatment, and the one or more patient characteristics comprise characteristics
(1) the cancer is right-sided colorectal cancer, and PD-L1 expression is positive on immune cells of the patient,
(2) the cancer is right-sided colorectal cancer, and PD-L1 expression is positive on tumor cells of the patient, or
(3) the cancer is colorectal cancer, and CMS of the patient is 1 or 4.

65. A method for treating a microsatellite stable (MSS) cancer patient, comprising a step of administering to the patient an effective amount of pembrolizumab and an effective amount of napabucasin or a prodrug thereof, or a pharmaceutically acceptable salt thereof, in combination, wherein the patient has one or more patient characteristics that indicate that the patient is responsive to the treatment, and the one or more patient characteristics comprise characteristics
(1) the cancer is right-sided colorectal cancer, and PD-L1 expression is positive on immune cells of the patient,
(2) the cancer is right-sided colorectal cancer, and PD-L1 expression is positive on tumor cells of the patient, or
(3) the cancer is colorectal cancer, and CMS of the patient is 1 or 4.

66. Use of a cancer stem cell inhibitor and an immune checkpoint inhibitor for the manufacture of a combination for treating a microsatellite stable (MSS) cancer patient, wherein the combination is administered to a patient having one or more patient characteristics that indicate that the patient is responsive to the treatment.

67. Use of a composition comprising a cancer stem cell inhibitor for the manufacture of a medicament for treating a microsatellite stable (MSS) cancer patient, wherein the composition is administered in combination with an immune checkpoint inhibitor to a patient having one or more patient characteristics that indicate that the patient is responsive to the treatment.

68. Use of a composition comprising an immune checkpoint inhibitor for the manufacture of a medicament for treating a microsatellite stable (MSS) cancer patient, wherein the composition is administered in combination with a cancer stem cell inhibitor to a patient having one or more patient characteristics that indicate that the patient is responsive to the treatment.

69. Use of a reactive oxygen species generator (ROS generator) and an immune checkpoint inhibitor for the manufacture of a combination for treating a microsatellite stable (MSS) cancer patient, wherein the combination is administered to a patient having one or more patient characteristics that indicate that the patient is responsive to the treatment.

70. Use of a composition comprising a reactive oxygen species generator (ROS generator) for treating a microsatellite stable (MSS) cancer patient, wherein the composition is administered in combination with an immune checkpoint inhibitor to a patient having one or more patient characteristics that indicate that the patient is responsive to the treatment.

71. Use of a composition comprising an immune checkpoint inhibitor for treating a microsatellite stable (MSS) cancer patient, wherein the composition is administered in combination with a reactive oxygen species generator (ROS generator) to a patient having one or more patient characteristics that indicate that the patient is responsive to the treatment.

72. A cancer stem cell inhibitor for use in treating a microsatellite stable (MSS) cancer patient, wherein a composition comprising the cancer stem cell inhibitor is administered in combination with an immune checkpoint inhibitor to a patient having one or more patient characteristics that indicate that the patient is responsive to the treatment.

73. An immune checkpoint inhibitor for use in treating a microsatellite stable (MSS) cancer patient, wherein a composition comprising the immune checkpoint inhibitor is administered in combination with a cancer stem cell inhibitor to a patient having one or more patient characteristics that indicate that the patient is responsive to the treatment.

74. A reactive oxygen species generator (ROS generator) for use in treating a microsatellite stable (MSS) cancer patient, wherein a composition comprising the reactive oxygen species generator (ROS generator) is administered in combination with an immune checkpoint inhibitor to a patient having one or more patient characteristics that indicate that the patient is responsive to the treatment.

75. An immune checkpoint inhibitor for use in treating a microsatellite stable (MSS) cancer patient, wherein a composition comprising the immune checkpoint inhibitor is administered in combination with a reactive oxygen species generator (ROS generator) to a patient having one or more patient characteristics that indicate that the patient is responsive to the treatment.

76. A method for treating a microsatellite stable (MSS) cancer patient, comprising a step of administering to the patient an effective amount of a reactive oxygen species generator (ROS generator) and an effective amount of an immune checkpoint inhibitor in combination, wherein the patient has one or more patient characteristics that indicate that the patient is responsive to the cancer treatment.

77. The method of Item 76, wherein the composition comprising the reactive oxygen species generator (ROS generator) is administered separately from the immune checkpoint inhibitor.

78. The method of Item 76, wherein the composition comprising the reactive oxygen species generator (ROS generator) is administered together with the immune checkpoint inhibitor.

### Effect or Invention

The present disclosure can provide effective cancer therapy for MSS cancer patients, in which therapeutic effects are difficult to appear.

### BRIEF DESCRIPTION OF DRAWINGS

FIG. 1 is a scheme showing outline of the clinical trial.

FIG. 2 is the Waterfall plot showing the tumor shrinkage effect of napabucasin and pembrolizumab combination therapy on MSS colorectal cancer patients. For the subjects enrolled in this study, chest, abdomen, and pelvic CT/MRI examinations were performed before the start of treatment and at 6, 12, 18, and 24 weeks after the start of treatment (and then every 9 weeks until the 42nd week, and every 12 weeks after the 42nd week) to evaluate the tumor shrinkage effect of the combination therapy. The tumor shrinkage effect was determined by a new response evaluation criteria in solid tumors (RECIST guideline) ver1.1 or irRECIST (immune-related response criteria) that applied RECIST v1.1 to the evaluation method of tumor shrinkage effect seen in immunotherapy. As a result, PR (Partial Response: defined as at least a 30% decrease in the sum of diameters of the target lesion, taking as reference the baseline sum diameter) was observed in 4 subjects with MSS colorectal cancer as the best overall response. The objective response rate (ORR) was 10%.

FIG. 3 shows the progression free survival (PFS) in MSS colorectal cancer patients. Of all the enrolled subjects, progression free survival (PFS) was evaluated in patients who received the dose recommended for the phase II part and had measurable lesions (FAS: Full Analysis Set). The PFS was defined as the period from the date of registration to the date of exacerbation or the date of death due to any cause, whichever came first. The survival function for the PFS was estimated using the Kaplan-Meier method. The median PFS for MSS colorectal cancer was 1.6 months.

FIG. 4 shows the overall survival (OS) in MSS colorectal cancer patients. The overall survival (OS) was evaluated in FAS. The OS was defined as the period from the date of registration to the date of death due to any cause. The OS was also analyzed in the same way as the PFS. As a result, the median OS for MSS colorectal cancer was 7.3 months.

FIG. 5 shows the frequency and grade of adverse events that occurred in subjects who received the napabucasin and pembrolizumab combination therapy. Adverse events (subjective and objective symptoms) were observed and recorded from the start of treatment to 30 days after the final administration of the protocol treatment. The adverse event was defined as any unfavorable or unintended sign (including abnormal laboratory test value), symptom or disease that occurred in subjects who received the study agents regardless of whether or not it had a causal relationship with the study agents. As the incidence of adverse event, the frequency of the worst grade according to CTCAE v4.03-JCOG in the whole course was calculated for each adverse event for the population who received this study treatment at least once (SP: Safety Population) among all registered patients. Main adverse events that occurred in more than 20% of SP in cohort A and cohort B were diarrhea, nausea, loss of appetite, and vomiting. The frequency and severity of adverse events were not significantly different from those with monotherapy of napabucasin or pembrolizumab, and this combination therapy was tolerable.

FIG. 6 shows the objective response rate (ORR) in the presence or absence of PD-L1 expression. From 40 MSS colorectal cancer subjects and 10 MSI-High colorectal cancer subjects who could submitted tumor specimens at baseline, sections having a thickness of 5 μm were cut from formalin-fixed paraffin-embedded (FFPE) tissue blocks and stained with PD-L1 IHC 22C3 pharmDx “Dako”. Of 5 MSS colorectal cancer subjects whose PD-L1 expression was 1% or higher on tumor cells, 4 had right-sided colorectal cancer and 1 had left-sided colorectal cancer. The ORRs of MSS right-sided and left-sided colorectal cancers that were positive for PD-L1 on tumor cells were 3/4 (75%) and 0/1 (0%), respectively. Of 29 MSS colorectal cancer subjects whose Combined Positive Score (CPS) (combined PD-L1 expression on tumor cells and immune cells) was 1% or more, 20 had right-sided colorectal cancer and 9 had left-sided colorectal cancer. The ORRs of MSS right-sided and left-sided colorectal cancers with CPS of 1% or more were 4/20 (20%) and 0/9 (0%), respectively. These results suggest that the characteristics “right-sided” and “PD-L1 positive” in MSS colorectal cancer patients can be useful factors to predict the therapeutic effect of this combination therapy.

FIG. 7 shows the objective response rate (ORR) or response (Clinical Benefit; defined as “partial response (PR)+stable disease (SD) of 4 months or more”) for colorectal cancer patients classified as CMS1 or CMS4. From subjects who could submitted tumor specimens at baseline, RNA was extracted from tumor tissues and gene expression was analyzed by RNA sequencing, and the subjects were classified into CMS1 to CMS4 according to the molecular subtype based on a classification method proposed by the Colorectal Cancer Subtyping Consortium (CRCSC) (Guinney J, et al.: Nat Med. 21(11): 1350-1356, 2015). Of 31 subjects that could submit tumor specimens at baseline, 5 were CMS1 (including 2 MSI colorectal cancer), 6 were CMS2, 4 were CMS3, 6 were CMS4, and 10 were unknown or unmeasurable. The ORR and Clinical Benefit of MSS colorectal cancer patients classified as CMS1 or CMS4 were 3/9 cases (33%) and 4/9 cases (44%), respectively, and the ORR and Clinical Benefit of MSS colorectal cancer patients classified as CMS 1 or 4 whose primary lesion was on the right side was 3/5 cases (60%) and 4/5 cases (80%), respectively. On the other hand, the ORR of MSS colorectal cancer patients classified as CMS2 and CMS3 were 0/6 cases (0%) and 1/4 cases (25%), respectively. One successful case with CMS3 had a mutation in the polymerase ε (POLE), a DNA polymerase involved in DNA replication and repair. Similar to MSI, POLE mutations indicate phenotypes with high mutation load in tumors and are therefore considered as promising predictive markers for immune checkpoint inhibitors, and successful cases of pembrolizumab monotherapy have been reported in colorectal cancer patients with POLE genetic mutations (Laetitia Nebot-Bral, et al. European Journal of Cancer 84 (2017) 290e303, J. Gong, et al, J. Natl. Compr. Canc. Netw. 15 (2) (2017) 142-147). From this, it was speculated that one responsive patient with CMS3 might also respond to pembrolizumab monotherapy. These results suggest that CMS1 or CMS4 can be a useful factor to predict the therapeutic effect of this combination therapy in patients with MSS colorectal cancer.

FIG. 8 is an exemplary schematic diagram of the system of the present disclosure.

FIG. 9 is an exemplary schematic diagram when the system of the present disclosure is physically separated, for example by using the cloud/a server.

### DESCRIPTION OF EMBODIMENTS

The present disclosure will be described hereinafter with reference to the best mode. Throughout the specification, it should be understood that expression with a singular form also encompasses a concept in its plural form, unless otherwise stated. Therefore, it should be understood that singular articles (e.g., “a”, “an”, “the” in English) also encompass a concept in its plural form unless otherwise stated. It should also be understood that the terms used herein are used in the meaning commonly used in the art unless otherwise stated. Thus, unless otherwise defined, all terminology and scientific terms used herein have the same meaning as commonly understood by one of ordinary skill in the art to which the present disclosure belongs. In case of conflict, the present specification (including definitions) takes precedence.

### (Definitions)

Definitions and/or basic technical contents of the terms particularly used in the present specification will be described below when appropriate.

As used herein, the term “biomarker” refers to a characteristic that is objectively measured and evaluated as an indicator of normal biological process, pathological process, or pharmacological response to therapeutic intervention.

As used herein, “patient characteristic” refers to any criterion by which patients can be stratified. Examples of patient characteristics used as selection criteria in the present disclosure include characteristics that the cancer is right-sided colorectal cancer; PD-L1 expression is positive in the patient (based on CPS score); PD-L1 expression is positive on tumor cells; PD-L1 expression is positive on immune cells; and CMS is 1 or 4.

Examples of immune cells include monocytes, neutrophils, eosinophils, basophils, T cells, B cells and NK cells. In the present specification, it is considered that a “biomarker” corresponding to a specific “patient characteristic” is present. Thus, by measuring a specific “biomarker” corresponding to a specific “patient characteristic”, the specific “patient characteristic” can be determined.

As used herein, “cancer” (“gan” written in hiragana or in Chinese character in Japanese interchangeably) is used to mean a malignant tumor that is highly atypical, proliferates faster than normal cells, and can infiltrate or metastasize to surrounding tissues destructively, or a condition in which such a malignant tumor is present. In the present disclosure, cancers include, but are not limited to, solid tumors and hematopoietic tumors.

As used herein, the term “microsatellite stable (MSS) cancer” refers to a cancer in which the number of microsatellite repeats in its tissue is not different from the number in a normal tissue, or a cancer that does not have any defect in mismatch repair (MMR)-related genes in its tissue. Microsatellite stability is determined by a PCR test for microsatellite instability (MSI) or an immunohistochemistry (IHC) test for mismatch repair (MMR)-related protein. The PCR test for MST testing can be performed by using a known test method covered by insurance (brand name: MSI testing kit (FARCO)). The IHC test is a known test method for confirming expression of mismatch repair proteins MLH1, MSH2, MSH6, and PMS2.

As used herein, being “responsive” (to a treatment) means that the treatment provides tumor regression or long-term suppression of growth, and it can judged, for example, by the criteria of Complete Response (CR), Partial Response (PR) and Stable Disease (SD) in RECIST guideline or irRECIST, which adapts the RECIST guideline to the evaluation method of tumor shrinkage effect seen in immunotherapies. For example, being responsive to the treatment with a combination of a cancer stem cell inhibitor and an immune checkpoint inhibitor, or a combination of a reactive oxygen species generator and an immune checkpoint inhibitor, is defined as being judged as Complete Response (CR) or Partial Response (PR) or being judged as maintaining Stable Disease (SD) for 4 months or more according to the RECIST guideline or ir RECIST within a certain period of time after the treatment of the combination. In one embodiment of the disclosure, it is predicted whether the patient is judged as Complete Response (CR) or Partial Response (PR) according to ir RECIST within a certain period of time after the treatment of the combination.

As used herein, the expression that PD-L1 expression is “positive” means that “PD-L1 expression is positive on immune cells of the patient” and/or “PD-L1 expression is positive on tumor cells of the patient”. For example, whether PD-L1 expression is positive can be determined based on the

Combined Positive Score (CPS), which is a score provided by combining PD-L1 expression of tumor cells and that of immune cells in a cancer tissue. If the score is 1% or more, it can be determined to be positive. In this case, it means that PD-L1 expression is observed in 1% or more of all surviving cells in the tumor tissue that includes tumor cells or immune cells. In some embodiments of the present disclosure, the proportion of PD-L1-expressing cells in either of tumor cells or immune cells in a cancer tissue may be used.

As used herein, the term “CMS” means a classification of colorectal cancer based on the consensus colorectal cancer molecular types by the Colorectal Cancer Subtyping Consortium (CRCSC). The CMS is determined by measuring gene expression in a tumor tissue by RNA sequencing or any other method and according to the subtype classification algorithm of Guinney et al. (Guinney J, et al.: Nat Med. 21(11):1350-1356, 2015).

### (Cancer Stem Cell Inhibitor)

The combination of medical agents in the present disclosure may comprise a cancer stem cell inhibitor. As used herein, the term “cancer stem cell inhibitor” refers to any substance that inhibits a property that makes a cell a cancer stem cell or a property possessed by a cancer stem cell. A cancer stem cell (CSC) has “self-renewal ability” and “pluripotency”, i.e., an ability to differentiate into many types of cells, and it is believed to, while maintaining the same cell as itself by self-renewal in cancer tissues, develop majority of surrounding cancer cells by differentiation. In addition, CSC has the ability to spread to other parts of the body by metastasis, after which CSC can give rise to new tumors (Jordan C T et al., N. Engl. J. Med. 2006; 355 (12): 1253-1261).

Examples of cancer stem cell inhibitors include a molecule that can target, reduce, inhibit, interfere with, or regulate at least one pathway that contributes a cancer stem cell property, or expression of at least one gene that contributes a cancer stem cell property (e.g., production of a functional product such as a protein). Examples of cancer stem cell biomarkers include, but are not limited to, β-catenin, NANOG, SMO, SOX2, STATS, AXL, ATM, c-MYC, KLF4, survivin, and BMI-1. These molecules are thought to be involved in cancer stem cell properties.

In certain embodiments, the cancer stem cell inhibitor is a small molecule that binds to a protein encoded by a cancer stem cell gene. In certain embodiments, the cancer stem cell inhibitor is a biologic such as a recombinant binding protein or peptide (e.g., APTSTAT3; see Kim et al., Cancer Res. (2014) 74(8): 2144-51) or a nucleic acid (e.g., STAT3 aiRNA; see U.S. Pat. No. 9,328,345 whose contents are incorporated herein by reference in its entirety) or a conjugate thereof that interacts with a cancer stem cell gene or a protein encoded by the gene. In certain embodiments, the cancer stem cell inhibitor is a cell. In certain embodiments, the cancer stem cell inhibitor is a STAT3 pathway inhibitor (e.g., a STAT3 inhibitor) (for example, an inhibitor that binds and inhibits the biological activity of STAT3, see Furtek et al., ACS Chem. Biol., 2016, 11(2), pp308-318).

As used herein, the term “STAT3 pathway inhibitor” refers to any molecule that inhibits any of the components of the STAT3 pathway, including STAT3 per se. The targeted protein inhibited by a STAT3 pathway inhibitor is not limited to STAT3 only.

In certain embodiments, the cancer stem cell inhibitor is at least one compound selected from 2-(1-hydroxyethyl)-naphtho[2,3-b]furan-4,9-dione, 2-acetyl-7-chloro-naphtho[2,3-b]furan-4,9-dione, 2-acetyl-7-fluoro-naphtho[2,3-b]furan-4,9-dione, 2-acetylnaphtho[2,3-b]furan-4,9-dione (napabcasin), and 2-ethyl-naphtho[2,3-b]furan-4,9-dione, and prodrugs, derivatives, or pharmaceutically acceptable salts thereof, and solvates thereof.

In one embodiment, the cancer stem cell inhibitor is a compound represented by Formula I:

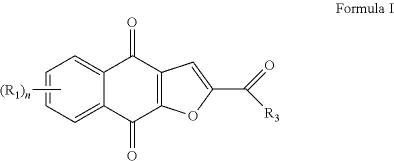

wherein,

each (R1) is independently selected from the group consisting of hydrogen, halogen, fluorine, cyano, nitro, CF₃, OCF₃, alkyl, methyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NH₂,

n is 0, 1, 2, 3, or 4, and

R₃ is selected from the group consisting of hydrogen, halogen, fluorine, cyano, CF₃, OCF₃, alkyl, methyl, substituted alkyl, halogen-substituted alkyl, hydroxyl-substituted alkyl, amine-substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NR_{b}R_{c}, wherein Rₐ is independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, and substituted aryl, and R_{b} and R_{c} are independently selected from the group consisting of hydrogen, alkyl, substituted alkyl, cycloalkyl, substituted cycloalkyl, heterocycle, substituted heterocycle, aryl, and substituted aryl, or R_{b} and R_{c} form a heterocycle or a substituted heterocycle together with the N to which they are attached, or a prodrug, derivative, pharmaceutically acceptable salt or solvate thereof.

In Formula I, the symbol (R₁)ₙ indicates that each available position along the benzene ring is independently substituted with a (R₁) substituent. For example, if n is equal to 4, all four R₁ substituents may be the same, or they may differ from each other. For example, each (R₁) can be independently selected from the group consisting of hydrogen, halogen, fluorine, cyano, nitro, CF₃, OCF₃, alkyl, methyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NH₂. Alkyls can include, for example, moieties having 1 to 8 carbon atoms linked by a single bond, and alkenyls can include, for example, moieties having 2 to 8 carbon atoms linked by one or more double bonds, and alkynyls can include, for example, moieties having 2 to 8 carbon atoms linked by one or more triple bonds. Substituents can include moieties such as hydrogen, halogen, cyano, nitro, aryl, ORₐ, SRₐ, and NH₂. For example, each (R₁) can be independently selected from the group consisting of hydrogen, methyl, F (fluorine), Cl (chlorine), Br (bromine), T (iodine), OH (hydroxyl), and NH₂ (amine). For example, R₃ can be selected from the group consisting of hydrogen, halogen, fluorine, cyano, CF₃, OCF₃, alkyl, methyl, substituted alkyl, halogen-substituted alkyl, hydroxyl-substituted alkyl, amine-substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NR_{b}R_{c}. For example, R₃ can be selected from the group consisting of methyl and C(R₈)₃. Each (R₈) can be independently selected from the group consisting of hydrogen, methyl, F (fluorine), Cl, Br, I, OH, and NH₂. For example, up to two of the independently selected (R₁) and (R₈) substituents can be selected to be F (fluorine) and the rest to be hydrogen.

In some embodiments, the compound of Formula I is selected from the group consisting of 2-(1-hydroxyethyl)-naphtho[2,3-b]furan-4,9-dione, 2-acetyl-7-chloro-naphtho[2,3-b]furan-4,9-dione, 2-acetyl-7-fluoro-naphtho[2,3-b]furan-4,9-dione, 2⁻acetylnaphtho[2,3-b]furan-4,9-dione, 2-ethyl-naphtho[2,3-b]furan-4,9-dione, and their enantiomers, diastereomers, tautomers, and salts or solvates thereof. For example, each (R₁) can be selected to be hydrogen and R₃ can be selected to be methyl so that the compound of Formula I is 2⁻acetylnaphtho[2,3-b]furan-4,9-dione. For example, each Rₐ can be independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, and substituted aryl. For example, each R_{b} and R_{c} can be independently selected from the group consisting of hydrogen, alkyl, substituted alkyl, cycloalkyl, substituted cycloalkyl, heterocycle, substituted heterocycle, aryl, and substituted aryl. Alternatively, R_{b} and R_{c} can form a heterocycle or a substituted heterocycle together with the N to which they are attached.

In one embodiment, the cancer stem cell inhibitor is a compound represented by the following formula:

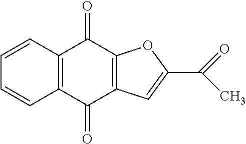 [SMILES: CC(=O)c1cc2c(o1)C(=O)c1ccccc1C2=O]

(napabucasin), or a prodrug, derivative, pharmaceutically acceptable salt or solvate thereof. The cancer stem cell inhibitor of the present disclosure may be any polymorph when present in crystalline form, and examples of polymorphs of the compound are described in WO2011/116399, for example.

In certain embodiments, the cancer stem cell inhibitor of the present disclosure can be administered in an amount of about 300 to about 700 mg. In certain embodiments, the cancer stem cell inhibitor can be administered in an amount of about 700 to about 1200 mg. In certain embodiments, the cancer stem cell inhibitor can be administered in an amount of about 800 to about 1100 mg. In certain embodiments, the cancer stem cell inhibitor can be administered in an amount of about 850 to about 1050 mg. In certain embodiments, the cancer stem cell inhibitor can be administered in an amount of about 960 to about 1000 mg.

In certain embodiments, the total amount of the cancer stem cell inhibitor is administered once daily. In certain embodiments, the cancer stem cell inhibitor is administered at a daily dose of about 480 mg. In certain embodiments, the cancer stem cell inhibitor is administered at a daily dose of about 960 mg. In certain embodiments, the cancer stem cell inhibitor is administered at a daily dose of about 1000 mg. Tn certain embodiments, the total amount of the cancer stem cell inhibitor is administered in divided doses greater than once daily, such as twice daily (BID) or more frequently. In certain embodiments, the cancer stem cell inhibitor is administered at a dose of about 240 mg twice daily. In certain embodiments, the cancer stem cell inhibitor is administered at a dose of about 480 mg twice daily. In certain embodiments, the cancer stem cell inhibitor is administered at a dose of about 500 mg twice daily. In certain embodiments, the cancer stem cell inhibitor is orally administered.

In one embodiment, the prodrug of napabucasin as a cancer stem cell inhibitor can be a compound of Formula 1A:

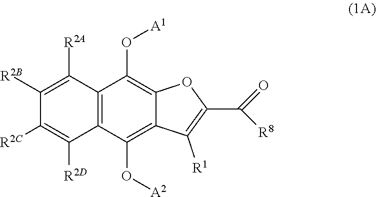

wherein A¹ and A² are the same or different and independently —C(═O)B, —CO₂B, —CONR^{3C}B, or a hydrogen atom, wherein A¹ and A² are not hydrogen atoms at the same time,

wherein B is

(1) a 3- to 6-membered monocyclic or polycyclic heterocyclic group,

(2) a 3- to 6-membered cyclic amino group, or

(3) a group represented by Formula (B):

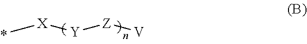

wherein the 3- to 6-membered monocyclic heterocyclic group and the 3- to 6-membered cyclic amino group have at least one secondary nitrogen atom in the ring, and

in Formula (B),

X is

(1) a single bond,

(2) C₁₋₆ alkylene, wherein the alkylene is optionally substituted with 1 to 3 substituents selected from the group consisting of a carboxyl group and —CO₂R⁶, or

(3) C₃₋₁₀ cycloalkylene,

Y is a single bond, an oxygen atom, or —NR^{4A}—,

R^{4A} is a hydrogen atom,

Z is

(1) a single bond or

(2) C₁₋₆ alkylene,

n is 0 or 1 and

V is

(1) —NHR⁵,

(2) a 3- to 6-membered monocyclic or polycyclic heterocyclic group, or

(3) a 3- to 6-membered cyclic amino group,

wherein the 3- to 6-membered monocyclic or polycyclic heterocyclic group and the 3- to 6-membered cyclic amino group have at least one secondary nitrogen atom in the ring,

R⁵ is

(1) a hydrogen atom or

(2) a C₁₋₆ alkyl group, wherein the alkyl group is optionally substituted with 1 to 3 substituents selected from the group consisting of a halogen atom, a hydroxyl group, a carboxyl group, a sulfinic acid group, a sulfonic acid group, a phosphoric acid group, a C₆₋₁₀ aryl group, a C₁₋₆ alkoxy group, a C₃₋₆ cycloalkoxy group, —NR⁶R⁷, —CO₂R⁶, —CONR⁶R⁷, —SO₂R⁶, —SO₂NR⁶R⁷, —OCO₂R⁶, —OCONR⁶R⁷ and —NR⁶CO₂R⁷, and

R⁶ and R⁷ are the same or different and independently a hydrogen atom or a C₁₋₆ alkyl group optionally substituted with 1 to 2 carboxyl groups, wherein, when both R⁶ and R⁷ are optionally substituted C₁₋₆ alkyl groups, they may form a 3- to 12-membered cyclic amino group together with the nitrogen atom to which they are attached, and

R^{3C} is a hydrogen atom;

R¹ is a hydrogen atom;

R^{2A}, R^{2B}, R^{2C} and R^{2D} are all hydrogen atoms;

R⁸ is a methyl group,

or a pharmaceutically acceptable salt or solvate thereof.

In one embodiment, the prodrug of napabucasin as a cancer stem cell inhibitor is a compound selected from the following compounds, or a pharmaceutically acceptable salt thereof:
2-acetylnaphtho[2,3-b]furan-4,9-diyl bis((3-aminopropyl)carbamate),
2-acetylnaphtho[2,3-b]furan-4,9-diyl bis((2-aminoethyl)carbamate),
(2S,2′S)-4,4′-((((2-acetylnaphtho[2,3-b]furan-4,9-diyl) bis(oxy))bis(carbonyl))bis(azanediyl))bis(2-(methylamino)butanoic acid),
2,2′-((((((2-acetylnaphtho[2,3-b]furan-4,9-diyl)bis(oxy))bis(carbonyl))bis(azanediyl))bis(ethane-2,1-diyl))bis(azanediyl)) diacetic acid,
3,3′-((((((2-acetylnaphtho[2,3-b]furan-4,9-diyl)bis(oxy))bis(carbonyl))bis(azanediyl))bis(ethane-2,1-diyl))bis(azanediyl))dipropionic acid,
(2S,2′S)-3,3′-((((2-acetylnaphtho[2,3-b]furan-4,9-diyl)bis(oxy))bis(carbonyl))bis(azanediyl))bis(2-aminopropionic acid),
dimethyl 4,4′-((((2-acetylnaphtho[2,3-b]furan-4,9-diyl)bis(oxy))bis(carbonyl))bis(azanediyl))(2S,2′S)-bis(2-aminobutanoate),
dimethyl 3,3′-((((2-acetylnaphtho[2,3-b]furan-4,9-diyl)bis(oxy))bis(carbonyl))bis(azanediyl))(2S,2′S)-bis(2-aminopropanoate),
2,2′-((((((2-acetylnaphtho[2,3-b]furan-4,9-diyl)bis(oxy))bis(carbonyl))bis(azanediyl))bis(propane-3,1-diyl))bis(azanediyl))diacetic acid,
2,2′-((2,2′-((((((2-acetylnaphtho[2,3-b]furan-4,9-diyl)bis(oxy))bis(carbonyl))bis(azanediyl))bis(ethane-2,1-diyl))bis(azanediyl))bis(acetyl))bis(azanediyl))diacetic acid,
2-acetylnaphtho[2,3-b]furan-4,9-diyl bis((2-((2-(piperidin-1-ylsulfonyl)ethyl)amino)ethyl)carbamate),
2-acetylnaphtho[2,3-b]furan-4,9-diyl bis((2-((2-(N,N-dimethylsulfamoyl)ethyl)amino)ethyl)carbamate),
2-acetylnaphtho[2,3-b]furan-4,9-diyl bis((2-((2-(isopropylsulfonyl)ethyl)amino)ethyl)carbamate),
2-acetylnaphtho[2,3-b]furan-4,9-diyl bis((3-((2-(N,N-dimethylsulfamoyl)ethyl)amino)propyl)carbamate),
2-acetylnaphtho[2,3-b]furan-4,9-diyl bis((3-((2-(methylsulfonyl)ethyl)amino)propyl)carbamate),
2-acetylnaphtho[2,3-b]furan-4,9-diyl bis((2-((2-(methylsulfonyl)ethyl)amino)ethyl)carbamate),
2-acetylnaphtho[2,3-b]furan-4,9-diyl bis((2-((2-(azetidin-1-yl)-2-oxoethyl)amino)ethyl)carbamate),
2-acetylnaphtho[2,3-b]furan-4,9-diyl bis((3-((2-(azetidin-1-yl)-2-oxoethyl)amino)propyl)carbamate),
2,2′-((2,2′-((((((2-acetylnaphtho[2,3-b]furan-4,9-diyl) bis(oxy))bis(carbonyl))bis(azanediyl))bis(propane-3,1-diyl))bis(azanediyl))bis(acetyl))bis(azanediyl))diacetic acid,
2-acetylnaphtho[2,3-b]furan-4,9-diyl bis((2-((2-(carbamoyloxy)ethyl)amino)ethyl)carbamate),
2-acetylnaphtho[2,3-b]furan-4,9-diyl bis((3-((2-(carbamoyloxy)ethyl)amino)propyl)carbamate),
2-acetylnaphtho[2,3-b]furan-4,9-diyl bis((2-((2-((methoxycarbonyl)amino)ethyl)amino)ethyl)carbamate),
2-acetylnaphtho[2,3-b]furan-4,9-diyl bis((3-((2-((methoxycarbonyl)amino)ethyl)amino)propyl)carbamate),
2-acetylnaphtho[2,3-b]furan-4,9-diyl bis((2-((2-((methoxycarbonyl)oxy)ethyl)amino)ethyl)carbamate),
2-acetylnaphtho[2,3-b]furan-4,9-diyl bis((3-((2-((methoxycarbonyl)oxy)ethyl)amino)propyl)carbamate),
2-acetyl-4-hydroxynaphtho[2,3-b]furan-9-yl (3-((2-(N,N-dimethylsulfamoyl)ethyl)amino)propyl)carbamate,
2-acetyl-4-hydroxynaphtho[2,3-b]furan-9-yl (3-((2-(methylsulfonyl)ethyl)amino)propyl)carbamate,
(3-((((2-acetyl-4-hydroxynaphtho[2,3-b]furan-9-yl)oxy)carbonyl)amino)propyl)glycylglycine,
2-acetyl-4-hydroxynaphtho[2,3-b]furan-9-yl (3-((2-(azetidin-1-yl)-2-oxoethyl)amino)propyl)carbamate,
2-acetyl-4-hydroxynaphtho[2,3-b]furan-9-yl (3-((2-(carbamoyloxy)ethyl)amino)propyl)carbamate,
2-acetyl-4-hydroxynaphtho[2,3-b]furan-9-yl (3-((2-((methoxycarbonyl)amino)ethyl)amino)propyl)carbamate, and
2-acetyl-4-hydroxynaphtho[2,3-b]furan-9-yl (3-((2-((methoxycarbonyl)oxy)ethyl)amino)propyl)carbamate.

### (Reactive Oxygen Species Generator (ROS Generator))

The combination of medical agents in the present disclosure may comprise a reactive oxygen species generator. As used herein, the term “reactive oxygen species generator” means an agent that increases intracellular reactive oxygen species such as superoxide, hydroxyl radicals, or hydrogen peroxide.

Specific examples of the reactive oxygen species generator include compounds that inhibit the removal of intracellular reactive oxygen species such as benzyl isothiocyanate, phenylethyl isothiocyanate, and butionine sulfoximin, or compounds having a quinone skeleton. More preferably, lapachol, Beta-lapachone, or napabucasin can be mentioned.

In certain embodiments, the reactive oxygen species generator is at least one compound selected from 2-(1-hydroxyethyl)-naphtho[2,3-b]furan-4,9-dione, 2-acetyl-7-chloro-naphtho[2,3-b]furan-4,9-dione, 2-acetyl-7-fluoro-naphtho[2,3-b]furan-4,9-dione, 2-acetylnaphtho[2,3-b]furan-4,9-dione (napabcasin), and 2-ethyl-naphtho[2,3-b]furan-4,9-dione, and prodrugs, derivatives, or pharmaceutically acceptable salts thereof, and solvates thereof.

In one embodiment, the reactive oxygen species generator is a compound represented by Formula I:

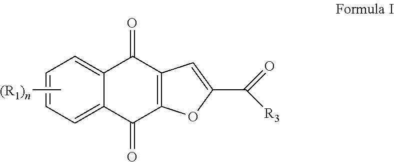

wherein,

each (R₁) is independently selected from the group consisting of hydrogen, halogen, fluorine, cyano, nitro, CF₃, OCF₃, alkyl, methyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NH₂,

n is 0, 1, 2, 3, or 4, and

R₃ is selected from the group consisting of hydrogen, halogen, fluorine, cyano, CF₃, OCF₃, alkyl, methyl, substituted alkyl, halogen-substituted alkyl, hydroxyl-substituted alkyl, amine-substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NR_{b}R_{c}, wherein Rₐ is independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, and substituted aryl, and R_{b} and R_{c} are independently selected from the group consisting of hydrogen, alkyl, substituted alkyl, cycloalkyl, substituted cycloalkyl, heterocycle, substituted heterocycle, aryl, and substituted aryl, or R_{b} and R_{c} form a heterocycle or a substituted heterocycle together with the N to which they are attached, or a prodrug, derivative, pharmaceutically acceptable salt or solvate thereof.

In Formula I, the symbol (R₁)ₙ indicates that each available position along the benzene ring is independently substituted with a (R₁) substituent. For example, if n is equal to 4, all four R₁ substituents may be the same, or they may differ from each other. For example, each (R₁) can be independently selected from the group consisting of hydrogen, halogen, fluorine, cyano, nitro, CF₃, OCF₃, alkyl, methyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NH₂. Alkyls can include, for example, moieties having 1 to 8 carbon atoms linked by a single bond, and alkenyls can include, for example, moieties having 2 to 8 carbon atoms linked by one or more double bonds, and alkynyls can include, for example, moieties having 2 to 8 carbon atoms linked by one or more triple bonds. Substituents can include moieties such as hydrogen, halogen, cyano, nitro, aryl, ORₐ, SRₐ, and NH₂. For example, each (R₁) can be independently selected from the group consisting of hydrogen, methyl, F (fluorine), Cl (chlorine), Br (bromine), I (iodine), OH (hydroxyl), and NH₂ (amine). For example, R₃ can be selected from the group consisting of hydrogen, halogen, fluorine, cyano, CF₃, OCF₃, alkyl, methyl, substituted alkyl, halogen-substituted alkyl, hydroxyl-substituted alkyl, amine-substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NR_{b}R_{c}. For example, R₃ can be selected from the group consisting of methyl and C(R₈)₃. Each (R₈) can be independently selected from the group consisting of hydrogen, methyl, F (fluorine), Cl, Br, I, OH, and NH₂. For example, up to two of the independently selected (R₁) and (R₈) substituents can be selected to be F (fluorine) and the rest to be hydrogen.

In some embodiments, the compound of Formula I is selected from the group consisting of 2-(1-hydroxyethyl)-naphtho[2,3-b]furan-4,9-dione, 2-acetyl-7-chloro-naphtho[2,3-b]furan-4,9-dione, 2-acetyl-7-fluoro-naphtho[2,3-b]furan-4,9-dione, 2⁻acetylnaphtho[2,3-b]furan-4,9-dione, 2-ethyl-naphtho[2,3-b]furan-4,9-dione, and their enantiomers, diastereomers, tautomers, and salts or solvates thereof. For example, each (R₁) can be selected to be hydrogen and R₃ can be selected to be methyl so that the compound of Formula I is 2-acetylnaphtho[2,3-b]furan-4,9-dione. For example, each Rₐ can be independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, and substituted aryl. For example, each R_{b} and R_{c} can be independently selected from the group consisting of hydrogen, alkyl, substituted alkyl, cycloalkyl, substituted cycloalkyl, heterocycle, substituted heterocycle, aryl, and substituted aryl. Alternatively, R_{b} and R_{c} can form a heterocycle or a substituted heterocycle together with the N to which they are attached.

In one embodiment, the reactive oxygen species generator is a compound represented by the following formula:

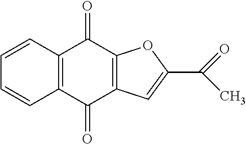 [SMILES: CC(=O)c1cc2c(o1)C(=O)c1ccccc1C2=O]

(napabucasin), or a prodrug, derivative, pharmaceutically acceptable salt or solvate thereof. The reactive oxygen species generator of the present disclosure may be any polymorph when present in crystalline form, and examples of polymorphs of the compound are described in WO2011/116399, for example.

In certain embodiments, the reactive oxygen species generator of the present disclosure can be administered in an amount of about 300 to about 700 mg. In certain embodiments, the reactive oxygen species generator can be administered in an amount of about 700 to about 1200 mg. In certain embodiments, the reactive oxygen species generator can be administered in an amount of about 800 to about 1100 mg. In certain embodiments, the reactive oxygen species generator can be administered in an amount of about 850 to about 1050 mg. In certain embodiments, the reactive oxygen species generator can be administered in an amount of about 960 to about 1000 mg.

In certain embodiments, the total amount of the reactive oxygen species generator is administered once daily. In certain embodiments, the reactive oxygen species generator is administered at a daily dose of about 480 mg. In certain embodiments, the reactive oxygen species generator is administered at a daily dose of about 960 mg. In certain embodiments, the reactive oxygen species generator is administered at a daily dose of about 1000 mg. In certain embodiments, the total amount of the reactive oxygen species generator is administered in divided doses greater than once daily, such as twice daily (BID) or more frequently. In certain embodiments, the reactive oxygen species generator is administered at a dose of about 240 mg twice daily. In certain embodiments, the reactive oxygen species generator is administered at a dose of about 480 mg twice daily. In certain embodiments, the reactive oxygen species generator is administered at a dose of about 500 mg twice daily. In certain embodiments, the reactive oxygen species generator is orally administered.

In one embodiment, the prodrug of napabucasin as a reactive oxygen species generator can be a compound of Formula 1A:

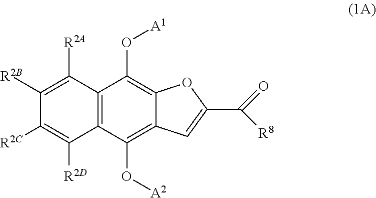

wherein A¹ and A² are the same or different and independently —C(═O)B, —CO₂B, —CONR^{3C}B, or a hydrogen atom, wherein A¹ and A² are not hydrogen atoms at the same time,

wherein B is

(1) a 3- to 6-membered monocyclic or polycyclic heterocyclic group,

(2) a 3- to 6-membered cyclic amino group, or

(3) a group represented by Formula (B):

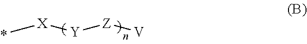

wherein the 3- to 6-membered monocyclic heterocyclic group and the 3- to 6-membered cyclic amino group have at least one secondary nitrogen atom in the ring, and

in Formula (B),

X is

(1) a single bond,

(2) C₁₋₆ alkylene, wherein the alkylene is optionally substituted with 1 to 3 substituents selected from the group consisting of a carboxyl group and —CO₂R⁶, or

(3) C3-10 cycloalkylene,

Y is a single bond, an oxygen atom, or —NR^{4A}—,

R⁴A is a hydrogen atom,

Z is

(1) a single bond or

(2) C₁₋₆ alkylene,

n is 0 or 1 and

V is

(1) —NHR⁵,

(2) a 3- to 6-membered monocyclic or polycyclic heterocyclic group, or

(3) a 3- to 6-membered cyclic amino group,

wherein the 3- to 6-membered monocyclic or polycyclic heterocyclic group and the 3- to 6-membered cyclic amino group have at least one secondary nitrogen atom in the ring,

R⁵ is

(1) a hydrogen atom or

(2) a C₁₋₆ alkyl group, wherein the alkyl group is optionally substituted with 1 to 3 substituents selected from the group consisting of a halogen atom, a hydroxyl group, a carboxyl group, a sulfinic acid group, a sulfonic acid group, a phosphoric acid group, a C₆₋₁₀ aryl group, a C₁₋₆ alkoxy group, a C₃₋₈ cycloalkoxy group, —NR⁶R⁷, —CO₂R⁶, —CONR⁶R⁷, —SO₂R⁶, —SO₂NR⁶R⁷, —OCO₂R⁶, —OCONR⁶R⁷ and —NR⁶CO₂R⁷, and

R⁶ and R⁷ are the same or different and independently a hydrogen atom or a C₁₋₆ alkyl group optionally substituted with 1 to 2 carboxyl groups, wherein, when both R⁶ and R⁷ are optionally substituted C₁₋₆ alkyl groups, they may form a 3- to 12-membered cyclic amino group together with the nitrogen atom to which they are attached, and

R^{3C} is a hydrogen atom;

R¹ is a hydrogen atom;

R^{2A}, R^{2B}, R^{2C} and R^{2D} are all hydrogen atoms;

R⁸ is a methyl group,

or a pharmaceutically acceptable salt or solvate thereof.

In one embodiment, the prodrug of napabucasin as a reactive oxygen species generator r is a compound selected from the following compounds, or a pharmaceutically acceptable salt thereof:
2-acetylnaphtho[2,3-b]furan-4,9-diyl bis((3-aminopropyl)carbamate),
2-acetylnaphtho[2,3-b]furan-4,9-diyl bis((2-aminoethyl)carbamate),
(2S,2′S)-4,4′-((((2-acetylnaphtho[2,3-b]furan-4,9-diyl) bis(oxy))bis(carbonyl))bis(azanediyl))bis(2-(methylamino)butanoic acid),
2,2′-((((((2-acetylnaphtho[2,3-b]furan-4,9-diyl)bis(oxy))bis(carbonyl))bis(azanediyl))bis(ethane-2,1-diyl))bis(azanediyl)) diacetic acid,
3,3′-((((((2-acetylnaphtho[2,3-b]furan-4,9-diyl)bis(oxy))bis(carbonyl))bis(azanediyl))bis(ethane-2,1-diyl))bis(azanediyl))dipropionic acid,
(2S,2′S)-3,3′-((((2-acetylnaphtho[2,3-b]furan-4,9-diyl)bis(oxy))bis(carbonyl))bis(azanediyl))bis(2-aminopropionic acid),
dimethyl 4,4′-((((2-acetylnaphtho[2,3-b]furan-4,9-diyl)bis(oxy))bis(carbonyl))bis(azanediyl))(2S,2′S)-bis(2-aminobutanoate),
dimethyl 3,3′-((((2-acetylnaphtho[2,3-b]furan-4,9-diyl)bis(oxy))bis(carbonyl))bis(azanediyl))(2S,2′S)-bis(2-aminopropanoate),
2,2′-((((((2-acetylnaphtho[2,3-b]furan-4,9-diyl)bis(oxy))bis(carbonyl))bis(azanediyl))bis(propane-3,1-diyl))bis(azanediyl))diacetic acid,
2,2′-((2,2′-((((((2-acetylnaphtho[2,3-b]furan-4,9-diyl)bis(oxy))bis(carbonyl))bis(azanediyl))bis(ethane-2,1-diyl))bis(azanediyl))bis(acetyl))bis(azanediyl))diacetic acid,
2-acetylnaphtho[2,3-b]furan-4,9-diyl bis((2-((2-(piperidin-1-ylsulfonyl)ethyl)amino)ethyl)carbamate),
2-acetylnaphtho[2,3-b]furan-4,9-diyl bis((2-((2-(N,N-dimethylsulfamoyl)ethyl)amino)ethyl)carbamate),
2-acetylnaphtho[2,3-b]furan-4,9-diyl bis((2-((2-(isopropylsulfonyl)ethyl)amino)ethyl)carbamate),
2-acetylnaphtho[2,3-b]furan-4,9-diyl bis((3-((2-(N,N-dimethylsulfamoyl)ethyl)amino)propyl)carbamate),
2-acetylnaphtho[2,3-b]furan-4,9-diyl bis((3-((2-(methylsulfonyl)ethyl)amino)propyl)carbamate),
2-acetylnaphtho[2,3-b]furan-4,9-diyl bis((2-((2-(methylsulfonyl)ethyl)amino)ethyl)carbamate),
2-acetylnaphtho[2,3-b]furan-4,9-diyl bis((2-((2-(azetidin-1-yl)-2-oxoethyl)amino)ethyl)carbamate),
2-acetylnaphtho[2,3-b]furan-4,9-diyl bis((3-((2-(azetidin-1-yl)-2-oxoethyl)amino)propyl)carbamate),
2,2′-((2,2′-((((((2-acetylnaphtho[2,3-b]furan-4,9-diyl) bis(oxy))bis(carbonyl))bis(azanediyl))bis(propane-3,1-diyl))bis(azanediyl))bis(acetyl))bis(azanediyl))diacetic acid,
2-acetylnaphtho[2,3-b]furan-4,9-diyl bis((2-((2-(carbamoyloxy)ethyl)amino)ethyl)carbamate),
2-acetylnaphtho[2,3-b]furan-4,9-diyl bis((3-((2-(carbamoyloxy)ethyl)amino)propyl)carbamate),
2-acetylnaphtho[2,3-b]furan-4,9-diyl bis((2-((2-((methoxycarbonyl)amino)ethyl)amino)ethyl)carbamate),
2-acetylnaphtho[2,3-b]furan-4,9-diyl bis((3-((2-((methoxycarbonyl)amino)ethyl)amino)propyl)carbamate),
2-acetylnaphtho[2,3-b]furan-4,9-diyl bis((2-((2-((methoxycarbonyl)oxy)ethyl)amino)ethyl)carbamate),
2-acetylnaphtho[2,3-b]furan-4,9-diyl bis((3-((2-((methoxycarbonyl)oxy)ethyl)amino)propyl)carbamate),
2-acetyl-4-hydroxynaphtho[2,3-b]furan-9-yl (3-((2-(N,N-dimethylsulfamoyl)ethyl)amino)propyl)carbamate,
2-acetyl-4-hydroxynaphtho[2,3-b]furan-9-yl (3-((2-(methylsulfonyl)ethyl)amino)propyl) carbamate,
(3-((((2-acetyl-4-hydroxynaphtho[2,3-b]furan-9-yl)oxy)carbonyl)amino)propyl)glycylglycine,
2-acetyl-4-hydroxynaphtho[2,3-b]furan-9-yl (3-((2-(azetidin-1-yl)-2-oxoethyl)amino)propyl)carbamate,
2-acetyl-4-hydroxynaphtho[2,3-b]furan-9-yl (3-((2-(carbamoyloxy)ethyl)amino)propyl)carbamate,
2-acetyl-4-hydroxynaphtho[2,3-b]furan-9-yl (3-((2-((methoxycarbonyl)amino)ethyl)amino)propyl)carbamate, and
2-acetyl-4-hydroxynaphtho[2,3-b]furan-9-yl (3-((2-((methoxycarbonyl)oxy)ethyl)amino)propyl)carbamate.

### (Immune Checkpoint Inhibitor)

The combination of medical agents in the present disclosure may comprise an immune checkpoint inhibitor. As used herein, an “immune checkpoint inhibitor” is any molecule capable of completely or partially inhibiting expression or function of one or more immune checkpoint molecules that control activation or function of T cells.

Non-limiting examples of immune checkpoint molecules include cytotoxic T lymphocyte-associated antigen (CTLA, e.g., CTLA4) and its ligands CD80 and CD86, programmed cell death protein (PD, e.g., PD-1) and its ligands PD-L1 and PD-L2, indolamine-pyrrole 2,3-dioxygenase-1 (IDO1), T cell membrane protein (TIM, e.g., TIM3), adenosine A2a receptor (A2aR), lymphocyte activation gene (LAG, e.g., LAG3), killer Immunoglobulin-like receptor (KIR), B7-H3, B7-H4, B7-H5 (VISTA), or TIGIT (T cell immunoreceptor with Ig and ITIM domain). These molecules are involved in co-stimulatory or inhibitory T cell responses. Immune checkpoint molecules regulate and maintain self-tolerance and persistence and magnitude of physiological immune responses. In certain embodiments, the immune checkpoint inhibitor can be a small molecule, antibody, nucleic acid, recombinant binding protein, or peptide that binds or inhibits the biological activity of an immune checkpoint molecule.

Immune checkpoint inhibitors include, but are not limited to, AMP-224, which is a recombinant B7-DC Fc fusion protein composed of the extracellular region of a PD-1 ligand, programmed cell death ligand 2 (PD-L2, B7-DC) and the Fc region of human immunoglobulin (Ig) G1 that binds to PD-1, and also called B7-DClg (see, for example, PCT/US2009/054969 and PCT/US2010/057940, the contents of which are incorporated herein by reference); atezolizumab, which binds to PD-L1, and also called MPDL3280A, RG7446, and YW243.55.S70 (see, for example, U.S. Pat. No. 8,217,149B, the contents of which are incorporated herein by reference); BMS-936559, which binds to Programmed Cell Death-1 Ligand (PD-L1), and also called MDX-1105 or 12A4 (see, for example, U.S. Pat. Nos. 7,943,743, 9,102,725, and 9,212,224, the contents of which are incorporated herein by reference); BMS-986016, which binds to LAG3 (CD223), and also called 25F7 or BMS 986016 (see, for example, US2015/0307609, the content of which is incorporated herein by reference); durvalumab, which binds to PD-L1, and also called MEDI-4736, MEDI4736 (see, for example, U.S. Pat. No. 8,779,108 and US2016/006,0344, the contents of which are incorporated herein by reference); IMP321, which is a 200 kDA soluble dimer recombinant fusion protein of the extracellular domain of LAG3 and immunoglobulin (see US2011/008331, the contents of which are incorporated herein by reference); ipilimumab, which binds to CTLA4, and also called Yervoy, MDX-010, MDX101, 10D1, BMS-734016 (see U.S. Pat. Nos. 6,984,720, 8,784,815, 8,685,394, the contents of which are incorporated herein by reference); lilylumab, which binds to killer immunoglobulin-like receptor (KIR), and also called IPH 2101, IPH2101, 1-7F9, IPH 2102, IPH2102, or BMS-986015 (see U.S. Pat. Nos. 8,119,775 and 8,981,065, the contents of which are incorporated herein by reference); enoblituzumab, which is a humanized mouse antibody that binds to B7-H3, and also called MGA271 (see, for example, U.S. Pat. Nos. 8,802,091 and 9,150,656, the contents of which are incorporated herein by reference); nivolumab, which binds to PD-1, and also called Opdivo, ONO-4538, MDX-1106, BMS-936558, 5C4 (see, for example, U.S. Pat. Nos. 8,008,449, 9,084,776, and 8,168,179, the contents of which are incorporated herein by reference); pembrolizumab, which binds to PD-1, and also called Keytruda, MK-3475, SCH 900475, or rambrolizumab (see, for example, U.S. Pat. Nos. 8,354,509, 9,220,776, 8,952,136, and 8,900,587, the contents of which are incorporated herein by reference); tremelimumab, which binds to CTLA4, and also called ticilimumab, CP-675206, clone 11.2.1 (see, for example, U.S. Pat. Nos. 6,682,736, 8,685,394, 7,824,679, and 8,143,379, the contents of which are incorporated herein by reference); and cemiplimab, which binds to PD-1, and also called REGN2810.

In certain embodiments, the immune checkpoint inhibitor is selected from an anti-PD-1 antibody (e.g., pembrolizumab, nivolumab or cemiplimab), an anti-PD-L1 antibody (e.g., atezolizumab (MPDL3280A), rambrolizumab (MK3475), durvalumab, or avelumab), and an anti-CTLA4 antibody (ipilimumab or tremelimumab (MEDI4736)).

In certain preferred embodiments, the immune checkpoint inhibitor is a PD-1/PD-L1 inhibitor (e.g., an anti-PD-1 antibody or an anti-PD-L1 antibody).

In certain embodiments, pembrolizumab is administered, for example, intravenously at a dose of about 2 mg/kg once every 3 weeks over a period of about 30 minutes. In certain embodiments, ipilimumab can be administered, for example, intravenously at a dose of about 3 mg/kg once every three weeks over a period of about 90 minutes, for a total of four doses. In certain embodiments, nivolumab is administered, for example, intravenously at a dose of about 3 mg/kg once every two weeks over a period of about 60 minutes.

### (Combination of Medical Agents)

In the combination of medical agents of the present disclosure, the two or more medical agents as defined may be formulated and administered as separate compositions or as a single composition. When administered as separate compositions, they may be administered at a time or at different times (but within the period in which the effect of the rest of the medical agents on the subject persists). In addition, these agents can be administered in different dosage forms and/or methods, respectively, for example, one compound can be administered topically and the other compound can be administered orally.

In one embodiment, the combination of the present disclosure may be administered in a 21-day cycle. The immune checkpoint inhibitor of the present disclosure may be administered on the first day of a 21-day cycle. The administration may be intravenous administration. The dose of the immune checkpoint inhibitor may be about 200 mg/body. In this 21-day cycle, the cancer stem cell inhibitor or reactive oxygen species generator of the present disclosure may be administered twice daily. The administration may be oral administration. The dose of the cancer stem cell inhibitor or reactive oxygen species generator may be about 480 mg at a time. In addition, the cancer stem cell inhibitor or reactive oxygen species generator may be administered alone for a certain period of time before the 21-day cycle. For example, the cycle can be started after 7 days of administration of the cancer stem cell inhibitor or reactive oxygen species generator alone.

### (Cancer)

The present disclosure provides a method for treating cancer with a particular combination of medical agents, or a composition or combination therefor. Cancers covered in this disclosure are, but not limited to, esophageal cancer, gastroesophageal junction cancer, renal cell cancer, lung cancer, gastrointestinal cancer, leukemia, lymphoma, myeloma, brain cancer, pancreatic cancer, endometrial cancer, prostate cancer, liver cancer, bladder cancer, gastroesophageal adenocarcinoma, chondrosarcoma, colorectal adenocarcinoma, colorectal cancer, breast cancer, renal cell cancer, ovarian cancer, head and neck cancer, melanoma, gastric adenocarcinoma, sarcoma, urogenital cancer, gynecological cancer, and adrenocortical cancer. In certain embodiments, the cancer is colorectal cancer. In certain embodiments, the cancer is colorectal adenocarcinoma. In certain embodiments, the cancer is melanoma. In certain embodiments, the cancer is breast cancer. In certain embodiments, the cancer is bladder cancer. In certain embodiments, the cancer is renal cell cancer. In certain embodiments, the cancer is pancreatic cancer. In certain embodiments, the cancer is endometrial cancer.

In certain embodiments, the cancer can be unresectable.

In certain embodiments, the cancer can be advanced. In certain embodiments, the cancer can be refractory. In certain embodiments, the cancer can be recurrent. In certain embodiments, the cancer can be metastatic.

The cancer patient means a patient suffering from the “cancer” as mentioned above.

One aspect of the disclosure provides the treatment for microsatellite stable cancer. The cancer can be cancer without mismatch repair deficiency. In particular, colorectal cancer is mentioned as the cancer in which microsatellite stability is concerned. In this disclosure, colorectal cancer being microsatellite stable and/or without mismatch repair deficiency may be targeted.

### (Microsatellite Stability (MSS))

A microsatellite refers to a place in genomic DNA where a short base sequence of about one to several bases is repeated. Microsatellite instability (MSI) is a phenomenon in which the number of microsatellite repeats in a tumor tissue is different from that in a non-tumor (normal) tissue due to decrease in function to repair base sequence errors that occur during DNA replication.

MSI/MSS can be determined by comparing the number of repeats of microsatellite sequences in genomic DNA of tumor and non-tumor tissues. For example, a region containing microsatellite repeats can be amplified by PCR and the number of repeats of microsatellite sequences can be compared. In comparison of a tumor tissue and a non-tumor tissue, if the number of repeats of the microsatellite marker in the tumor tissue is changed, it is judged to be positive for microsatellite instability (MSI), and if it is not changed, it is judged to be microsatellite stable (MSS). Panels of microsatellite markers have been developed for microsatellite stability testing, and Bethesda panels (BAT25, BAT26, D5S346, D2S123 and D17S250) can be used, for example. When the 5 types of markers (Bethesda panel) is used, it can be judged as high-frequency microsatellite instability (MSI-H) if 2 or more markers show MSI; low-frequency microsatellite instability (MSI-L) if MSI is found in one place; microsatellite stable (MSS) if no MSI is observed. When a panel of other markers is used for MSI testing, it can be judged as MSI-H if 30 to 40% or more of all markers shows MSI, MSI-L if less markers show MSI, and MSS if MSI is not observed in any markers, for example.

In MSI-H colorectal cancer, 10 to 100 times more somatic mutations occur than in MSS colorectal cancer due to decrease in function of DNA mismatch repair genes. It is considered that the antigens derived from these gene mutations (neoantigens) are recognized as non-self, and thus immune response in the tumor tissue is enhanced, and expression of various immune checkpoint molecules is induced as negative feedback. The proportion of colorectal cancer patients with high-frequency microsatellite instability is said to be about 14 to 16%, and 5 to 6% of which are predicted to be stage 4 colorectal cancer patients. The frequency of high-frequency microsatellite instability is said to be higher in those who develop colorectal cancer at early ages. In addition to colorectal cancer, it is known that high-frequency microsatellite instability is common in endometrial cancer, gastric cancer, and small intestine cancer.

It has been revealed that anti-cancer agents including immune checkpoint inhibitors tend to be more effective against types of cancers with many genetic damages. They are often effective in cancer with deficiency in the mismatch repair mechanism or with microsatellite instability, but on the other hand, often ineffective against microsatellite stable cancer. The present disclosure can provide the treatment of microsatellite stable cancer with a particular combination of medical agents. In addition, selecting patients based on one or more patient characteristics can provide good clinical response even in patients with microsatellite stable cancer.

### (Patient Characteristics)

As used herein, a “patient characteristic” refers to a criterion by which patients can be stratified. Patient characteristics used as selection criteria in this disclosure include, for example, characteristics that the cancer is right-sided colorectal cancer; PD-L1 expression is positive in the patient (based on CPS score); PD-L1 expression is positive on tumor cells; PD-L1 expression is positive on immune cells; and CMS is 1 or 4.

The expression “PD-L1 expression is positive” as used herein include a case where “PD-L1 expression is positive on immune cells of the patient” and/or a case where “PD-L1 expression is positive on tumor cells of the patient”. Of the patient characteristics described herein, one or more patient characteristics may be used in combination, or other patient characteristics known in the art may be used in combination.

Examples of combinations of patient characteristics include “the cancer is right-sided colorectal cancer and PD-L1 expression is positive” and/or “the cancer is colorectal cancer and CMS is 1 or 4”.

Examples of immune cells include monocytes, neutrophils, eosinophils, basophils, T cells, B cells and NK cells. PD-L1 expression can be determined based on a positive CPS score, but in some embodiments, PD-L1 expression may be measured for tumor cells and may be determined based on a positive PD-L1 expression (1% or more) on tumor cells.

CMS is determined by the subtype classification algorithm of Guinney et al. by measuring gene expression in a tumor tissue by RNA sequencing or other techniques (Guinney J, et al.: Nat Med. 21(11):1350-1356, 2015). CMS refers to the classification of colorectal cancer based on the consensus colorectal cancer molecular types by the Colorectal Cancer Subtyping Consortium (CRCSC). It can be classified into CMS1 to CMS4 based on the gene expression pattern.

CMS1 has many gene mutations and it is characterized by a phenotype in which the immune system is activated. CMS2 is epithelial and characterized by a phenotype in which the WNT and MYC signaling pathways are activated. CMS3 is epithelial and characterized by a phenotype associated with metabolic disorders. CMS4 is mesenchymal and characterized by a phenotype associated with epithelial-mesenchymal transition, cancer stem cells and angiogenesis. As a patient characteristic, CMS of 1 (the fact that colorectal cancer is classified as CMS1) can be used. As a patient characteristic, CMS of 2 (the fact that colorectal cancer is classified as CMS2) can be used. As a patient characteristic, CMS of 3 (the fact that colorectal cancer is classified as CMS3) can be used. As a patient characteristic, CMS of 4 (the fact that colorectal cancer is classified as CMS4) can be used.

As a patient characteristic, one or more gene mutations in a patient can be used. For each gene mutation, its presence or absence can be used. For example, the presence or absence of an EGFR gene mutation, the presence or absence of an ALK fusion gene, or the presence or absence of a gene mutation of polymerase ε (POLE) can be used. In an example, the absence of a gene mutation in polymerase s (POLE) can be used as a patient characteristic. In another example, the presence of a gene mutation in polymerase s (POLE) can be used as a patient characteristic. The presence or absence of a gene mutation can be used in combination with other patient characteristics, if desired.

The large intestine is the gastrointestinal tract composed of the cecum, ascending colon, transverse colon, descending colon, sigmoid colon, and rectum. As used herein, the term “right-sided colorectal (rectal and colon) cancer” refers to cancer in which the right side of the large intestine (the cecum, ascending colon, and transverse colon) is the primary site, and the term “left-sided colorectal (rectal and colon) cancer” refers to cancer in which the left side of the large intestine (descending colon, sigmoid colon and rectum) is the primary site. The right-sided colorectal cancer is less likely to show symptoms until it grows larger, is often found as a tumor mass, and has anemia due to chronic bleeding. On the other hand, the left-sided colorectal cancer is often diagnosed by symptoms such as bleeding (melena, mucous stool), constipation/diarrhea, and thinning of stool. Although it is not intended to be bound by any specific theory, the laterality is considered to be caused by the facts that the right (proximal) large intestine and the left (distal) large intestine are developmentally different as the former is from the middle intestine and the latter is from the posterior intestine; the right-sided large intestine is controlled by the superior mesenteric artery system and the left-sided large intestine is controlled by the inferior mesenteric artery system; and the mucosal structures differ between the ascending colon and the descending colon. In addition, the composition of intestinal bacteria (flora) may be different on the left and right sides of the large intestine, and the right-sided colorectal cancer may be often caused by intestinal bacteria or inflammation.

### (Diagnostic Method)

An embodiment of the present disclosure is a method comprising using one or more patient characteristics in a microsatellite stable (MSS) cancer patient as an indicator of responsiveness of the patient to a cancer treatment. The cancer treatment may include combination use of a cancer stem cell inhibitor and an immune checkpoint inhibitor. The presence and/or absence of the one or more patient characteristics may indicate that the patient is responsive and/or not responsive to a cancer treatment. The one or more patient characteristics include characteristics that the cancer is right-sided colorectal cancer, PD-L1 expression is positive in the patient (based on CPS score), and PD-L1 expression is positive on tumor cells of the patient, PD-L1 expression is positive on immune cells of the patient, and CMS is 1 or 4. Of the patient characteristics described herein, one or more patient characteristics may be used in combination, or any other patient characteristic known in the art may be used in combination. Examples of combinations of patient characteristics include “the cancer is right-sided colorectal cancer and PD-L1 expression is positive” or “the cancer is colorectal cancer and CMS is 1 or 4”.

In one embodiment of the present disclosure, one or more patient characteristics in a cancer patient are used as an indicator to predict whether or not the patient will show complete or partial response to a cancer treatment. In one embodiment of the present disclosure, one or more patient characteristics in a cancer patient are used as an indicator to predict whether or not the patient will show complete or partial response to a cancer treatment, or will maintain stable disease for 4 months or longer in response to a cancer treatment. In one embodiment of the present disclosure, it may be determined whether or not the cancer treatment should be given based on the prediction result.

### (Diagnostic Agent/Kit)

An embodiment of the present disclosure is a composition for determining responsiveness of a microsatellite stable (MSS) cancer patient to a cancer treatment comprising combination use of a cancer stem cell inhibitor or ROS generator and an immune checkpoint inhibitor, the composition comprising a detection agent for PD-L1. A characteristic that PD-L1 expression is positive in the patient (based on CPS score); PD-L1 expression is positive on tumor cells of the patient; or PD-L1 expression is positive on immune cells of the patient can indicate that the patient is responsive to the cancer treatment. Examples of the detection agent include, but are not limited to, anti-PD-L1 antibodies. A kit comprising such a composition can also be provided. The kit may comprise a package insert or label provided to instruct physicians and other healthcare professionals to identify the patient as mentioned above as a subject to be treated. Not only by these official documents, treatment guidelines can be given to physicians or others by different paper media or from other information sources via the Internet.

### (System)

In a further embodiment of the present disclosure, a system for determining responsiveness of a patient to a cancer treatment can be provided. The system may comprise a storage for storing patient characteristic information of a plurality of patients and responsiveness information of the plurality of individuals, a learning unit configured to learn relationship between a patient characteristic and responsiveness from the patient characteristic information and the responsiveness information of the plurality of individuals, a processor that predicts responsiveness of a patient from a patient characteristic of the patient based on the relationship between the patient characteristic and responsiveness. As used herein, the term “system” refers to a configuration for performing the method or program of the present disclosure, and the term originally means a combination or organization for accomplishing an object, and a plurality of elements are systematically configured and affect each other. In the field of computers, it refers to the entire configuration including hardware, software, OS, and network. If necessary, the system may further comprise a display that displays the responsiveness information predicted by the processor. The present disclosure can also provide a program or a method that realizes the above system, or a recording medium comprising the same. In one embodiment, the learning is performed using Artificial Intelligence (AI).

The storage may be a recording medium stored in or detached from the system, such as a CD-R, DVD, Blueray, USB, SSD, or hard disk, or may be stored in a server or appropriately recorded on the cloud storage.

The machine learning can use linear regression, logistic regression, or support vector machine, and the discrimination accuracy of each model can be evaluated by cross-validation (CV). After ranking, the features can be increased one by one, and machine learning (such as linear regression, logistic regression, support vector machine) and cross-validation can be performed to evaluate the discrimination accuracy of each model. Then, the model with the highest accuracy can be selected. In the present disclosure, any machine learning can be used, and linear, logistic, support vector machine (SVM) can be used for supervised machine learning.

In addition, the system can distribute its functions to patients' terminals or terminals arranged in hospitals, for example. In one example, the input is made through a patient's information terminal or an information terminal in a hospital, for example (such as a mobile phone or smartphone). The input patient characteristics may be transmitted via the Internet and applied to a prediction model on the cloud service, and the prediction result may be transmitted to the information terminal.

The present disclosure also provides a program, program product, software, or application that implements the method for determining responsiveness of a patient to the cancer treatment with the medical agent or the combination of medical agents provided in this disclosure that is stored or mediated to function in a mobile terminal such as a smartphone or smartwatch.

The method for determining responsiveness of a patient to a cancer treatment according to the present disclosure comprises: A) providing patient characteristic information of a plurality of patients and responsiveness information of the plurality of individuals to a computer, B) making the computer learn the relationship between a patient characteristic and responsiveness from the patient characteristic information and the responsiveness information of the plurality of individuals, C) making the computer predict responsiveness of a patient from a patient characteristic of the patient based on the relationship between the patient characteristic and responsiveness, and D) displaying a prediction result on a display of a mobile terminal as needed.

In the present disclosure, the information in A) may be stored in a recording medium detached from or mounted in the computer, may be provided when required, for example, on the Internet, or may be stored in the cloud storage. The patient characteristic information and the responsiveness information may be stored in the same place or in different places. There are various methods that make a computer learn the relationship between a patient characteristic and responsiveness. A discriminant model may be created by machine learning.

The following is an example of a method for creating a discriminant model. By machine learning, a discriminant model for the relationship between a patient characteristic and responsiveness is created (determination of a coefficient by Lasso and Bayesian optimization). The generalization capability of the model is checked by using other samples. A “sample amplification method” may be used. The “sample amplification method” is a technique for significantly increasing the number of samples by using the distribution characteristics even when the number of samples is small. As an example of model creation, using a large number of samples as training data, a hyperparameter (λ) of logistic regression model can be determined by Lasso and Bayesian optimization, and the weighting coefficients of the features and the model intercept can be determined. As a large number of samples, if necessary, an amplified one with normal random numbers and Pearson system random numbers can be used.

The created model can be subject to model evaluation. That is, the model is used to discriminate and estimate other sample test data.

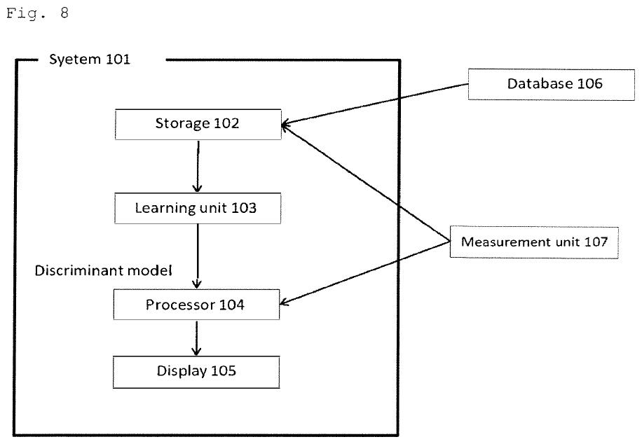

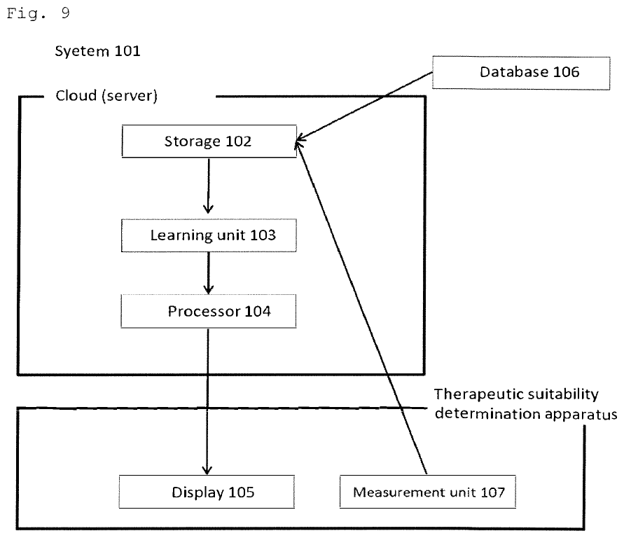

The therapeutic suitability determination technology of the present disclosure may be provided as a single system or apparatus including all elements (FIG. 7). Alternatively, it can also be considered that a therapeutic suitability determination apparatus mainly measures a patient characteristic and responsiveness and displays the results, and calculation or calculation of the discriminant model is performed on a server or the cloud service (FIG. 8). Some or all of these can be implemented using the Internet of Things (IoT) and/or Artificial Intelligence (AI).

Alternatively, the therapeutic suitability determination technology can be a semi-standalone type, that is, it may also store the discriminant model and performs discrimination on site, but the main calculation such as calculation of the discriminant model is performed on a server or the cloud service. It is a model that can be used even when shielded, since transmission and reception are not always possible in some places such as hospitals.

Therefore, in one aspect, the present disclosure provides a program to make a computer perform a method for determining therapeutic suitability of a subject, and a recording medium, system and apparatus in which the program is stored, wherein the method comprises: A) providing patient characteristic information of a plurality of patients and responsiveness information of the plurality of individuals to a computer, B) making the computer learn the relationship between a patient characteristic and responsiveness from the patient characteristic information and the responsiveness information of the plurality of individuals, C) making the computer predict responsiveness of a patient from a patient characteristic of the patient based on the relationship between the patient characteristic and responsiveness, and D) displaying a prediction result on a display of a mobile terminal as needed. A system that executes such a program may be realized in an embodiment in which the whole is regarded as a system.

The visualizer (display) may be any product as long as the user can recognize the therapeutic suitability determination result of a subject through it, and an input/output device, display device, television, or monitor may be used. Instead of the visualizer, other recognition means such as voice recognition may be used, and a sound generator (e.g., speaker), vibrating device, electrode, or other devices capable of presenting a challenge to the test subject may be included.

The recording medium may be, for example, a recording medium such as a CD-R, DVD, Blueray, USB, SSD, or hard disk, and may be stored in a server or appropriately recorded on the cloud storage.

Generally, “Software as a service (SaaS)” corresponds to such a cloud service. Since early therapeutic suitability determination technologies are considered to comprise discrimination algorithms created from data in a laboratory environment as one embodiment, the system may be provided as a system with a feature including two or three such embodiments.

### (Pharmaceutical Formulation)

The cancer stem cell inhibitor or reactive oxygen species generator and/or immune checkpoint inhibitor disclosed herein can be in the form of a pharmaceutical composition. In certain embodiments, the pharmaceutical composition may comprise at least one cancer stem cell inhibitor. In certain embodiments, the pharmaceutical composition may comprise at least one compound of Formula I and at least one pharmaceutically acceptable carrier. In certain embodiments, the pharmaceutical composition may comprise at least one immune checkpoint inhibitor. In certain embodiments, the pharmaceutical composition may comprise one or more compounds and at least one pharmaceutically acceptable carrier, wherein the one or more compounds can be converted to at least one compound of Formula I in a subject (i.e., can be prodrugs of the same). In certain embodiments, the pharmaceutical composition may comprise one or more compounds and at least one pharmaceutically acceptable carrier, wherein the one or more compounds can be converted to at least one immune checkpoint inhibiter in a subject (i.e., can be prodrugs of the same).

The plurality of agents described in the present disclosure may be comprised in a single composition (mixture) or in separate compositions. When formulated as a single composition, an immune checkpoint inhibitor and a cancer stem cell inhibitor or reactive oxygen species generator or a prodrug thereof may be comprised in a formulation in any of the forms known in the art including those exemplified herein.

As used herein, the term “carrier” refers to a pharmaceutically acceptable substance, composition, or excipient that relates to, or enables, for example, transporting or transferring a medical compound of interest from one organ or part of the body to another organ or part of the body, such as a liquid or solid bulking agent, diluent, additive, solvent, or encapsulating agent. The term “pharmaceutically acceptable” means that it is compatible with other ingredients in formulations and is not harmful to patients. Non-limiting examples of pharmaceutically acceptable carriers, carriers, and/or diluents include sugars such as lactose, glucose, and sucrose, starches such as corn starch and potato starch, cellulose and its derivatives such as sodium carboxymethyl cellulose, ethyl cellulose, cellulose acetate, excipients such as powdered tragacanth, malt, gelatin, talc, cacao butter and suppository wax, oils such as peanut oil, cottonseed oil, safflower oil, sesame oil, olive oil, corn oil, and soybean oil, glycols such as propylene glycol, polyols such as glycerin, sorbitol, mannitol, and polyethylene glycol, esters such as ethyl oleate and ethyl laurate, buffers such as agar, magnesium hydroxide and aluminum hydroxide, alginic acid, water without pyrogeneous substances, isotonic physiological saline, Ringer's solution, ethyl alcohol, phosphate buffers, and other non-toxic compatible substances used in pharmaceutical formulations. The composition may also comprise wetting agents, emulsifiers, and lubricants such as sodium lauryl sulfate, magnesium stearate, and polyethylene oxide-polypropylene oxide copolymers, as well as colorants, releasing agents, coating agents, sweeteners, flavors and seasonings, preservatives, and antioxidants.

The composition disclosed herein being suitable for oral administration can be a capsule, cachet, pill, tablet, rhombic tablet (usually using flavor bases that are sucrose and acacia or tragacanth), powder, granule, aqueous or non-aqueous liquid solution, aqueous or non-aqueous liquid suspension, oil-in-water emulsion, water-in-oil emulsion, elixir, syrup, lozenge (using inert bases such as gelatin, glycerin, sucrose, and/or acacia), and/or a dosage form of mouthwash, each comprising a predetermined amount of at least one compound of the present disclosure.

The composition disclosed herein can be administered as bolus, electuary, or paste.

A solid dosage form for oral administration (such as capsule, tablet, pill, sugar-coated tablet, powder, granule) can be mixed with one or more pharmaceutically acceptable carriers such as sodium citrate or dicalcium phosphate, and/or any of fillers or bulking agents such as starch, lactose, sucrose, glucose, mannitol, and/or silicic acid, binders such as carboxymethyl cellulose, alginate, gelatin, polyvinylpyrrolidone, sucrose, and/or acacia, moisturizers such as glycerol, disintegrants such as agar, calcium carbonate, potato or tapioca starch, alginic acid, certain silicates, sodium carbonate and sodium glycolic acid starch, dissolution retarders such as paraffin, absorption enhancers such as quaternary ammonium compounds, wetting agents such as cetyl alcohol, glycerol monostearate, and polyethylene oxide-polypropylene oxide copolymers, absorbents such as kaolin and bentonite clay, lubricants such as talc, calcium stearate, magnesium stearate, solid polyethylene glycol, sodium lauryl sulfate, and mixtures thereof, and colorants. For capsules, tablets, and pills, the pharmaceutical composition may also comprise a buffering agent. Similar types of solid compositions can also be used as fillers in soft and hard filled gelatin capsules with additives such as lactose or sugar of milk and a high molecular weight polyethylene glycol.

Liquid dosage forms for oral administration may include pharmaceutically acceptable emulsion, microemulsion, solution, suspension, syrup, and elixir. In addition to the active ingredient, the liquid dosage form can comprise an inert diluent used in the art, such as water or any other solvent, solubilizer, or emulsifier, and ethyl alcohol, isopropyl alcohol, ethyl carbonate, ethyl acetate, benzyl alcohol, benzyl benzoate, propylene glycol, 1,3-butylene glycol, oils (especially cottonseed oil, peanut oil, corn oil, germ oil, olive oil, castor oil, and sesame oil), glycerol, tetrahydrofuryl alcohol, polyethylene glycol, fatty acid esters of sorbitan, and mixtures thereof can be mentioned. In addition, cyclodextrins such as hydroxypropyl-β-cyclodextrin can be used to dissolve compounds.

The composition can also comprise an auxiliary such as a wetting agent, emulsifying and suspending agent, sweetener, seasoning, colorant, flavor, or preservative. Suspensions can comprise a suspending agent in addition to one or more compounds according to the present disclosure, and ethoxylated isostearyl alcohol, polyoxyethylene sorbitol and sorbitan ester, microcrystalline cellulose, aluminum metahydroxydoe, bentonite, agar, and tragacanth, as well as mixtures thereof, can be mentioned.

The composition disclosed herein can be a suppository for rectal or vaginal administration. It can be prepared by mixing one or more compounds according to the present disclosure with one or more suitable non-irritating additives or carriers such as cocoa butter, polyethylene glycol, suppository wax, and salicylate. It is solid at room temperature but liquid at body temperature and thus melts in the rectum or vaginal cavity to release the compound of the present disclosure. Pharmaceutical compositions suitable for vaginal administration may also include pessary, tampon, cream, gel, paste, foam, and spray formulation comprising a carrier known to be suitable in the prior art.

Dosage forms for topical or transdermal administration of the composition of the present disclosure can include powder, spray, ointment, paste, cream, lotion, gel, solution, patch, and inhalant. The pharmaceutical composition or tablet can be mixed under sterile conditions with a pharmaceutically acceptable carrier and, if required, a preservative, buffer, or bottled gas.

The ointment, paste, cream, and gel can comprise, in addition to the composition of the present disclosure, an additive such as an animal or vegetable fat, oil, wax, paraffin, starch, tragacanth, cellulose derivative, polyethylene glycol, silicone, bentonite, silicic acid, talc, or zinc oxide or a mixture thereof.

The powder and spray can comprise, in addition to the pharmaceutical composition or tablet of the present disclosure, an additive such as lactose, talc, silicic acid, aluminum hydroxide, calcium silicate, or polyamide powder, or a mixture thereof. In addition, the spray can comprise a common high pressure gas such as chlorofluorohydrocarbon, as well as volatile unsubstituted hydrocarbon such as butane or propane. Ophthalmic preparations, ophthalmic ointment, powder, and solution are also construed as being within the scope of the present disclosure.

Compositions suitable for parenteral administration can include at least one pharmaceutically acceptable sterile isotonic aqueous or non-aqueous solution, dispersion, suspension, emulsion, or sterile powder that can be reconstituted into a sterile injectable solution or dispersion just before use.

As used herein, the term “salt” includes acids and/or basic salts formed by inorganic and/or organic acids and bases. As used herein, the term “pharmaceutically acceptable salt” means a salt that is suitable for use in contact with subjects' tissues without undue toxicity, irritation, allergic reactions, and/or similar events and has a balanced legitimate effect/risk ratio within secure medical judgments. Pharmaceutically acceptable salts are well known in the art. For example, Berger et al. describe in detail pharmaceutically acceptable salts in J. Pharmaceutical Sciences (1977) 66: 1-19.

A pharmaceutically acceptable salt can be produced by an inorganic or organic acid. Non-limiting examples of suitable inorganic acids include hydrochloric acid, hydrobromic acid, phosphoric acid, sulfuric acid, and perchloric acid. Non-limiting examples of suitable organic acids include acetic acid, oxalic acid, maleic acid, tartaric acid, citric acid, succinic acid, and malonic acid. Other non-limiting examples of suitable pharmaceutically acceptable salts include adipate, alginate, ascorbate, asparaginate, benzenesulfonate, besilate, benzoate, bisulfate, borate, butyrate, camphorate, camphorsulfonate, citrate, cyclopentanepropionate, digluconate, dodecyl sulfate, ethanesulfonate, formate, fumarate, glucoheptonate, glycerophosphate, gluconate, hemisulfate, heptaneate, hexanate, hydroiodate, 2-hydroxy-ethanesulfonate, lactobionate, lactate, laurate, laurylsulfate, malete, maleate, malonate, methanesulfonate, 2-naphthalene sulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate. In some embodiments, the organic acid that can produce a salt include, for example, acetic acid, propionic acid, glycolic acid, pyruvic acid, oxalic acid, lactic acid, trifluolacetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid.

A salt can be prepared in-situ during separation and purification of the disclosed compound, or separately, for example by reacting the compound with an appropriate base or acid, respectively. Non-limiting examples of pharmaceutically acceptable salts obtained from bases include alkali metal, alkaline earth metal, ammonium, and N⁺(C1-4 alkyl)₄ salts. Non-limiting examples of suitable alkali or alkaline earth metal salts include sodium, lithium, potassium, calcium, magnesium, iron, zinc, copper, manganese, and aluminum salts. In addition, non-limiting examples of suitable pharmaceutically acceptable salts include salts obtained from non-toxic ammonium, quaternary ammonium, as well as amine cations formed using counter ions such as halide ion, hydroxide ion, carboxylate ion, sulfate ion, phosphate ion, nitrate ion, lower alkyl sulfonic acid ion, and aryl sulfonic acid ion. Non-limiting examples of suitable organic bases that can give rise to salts include primary amine, secondary amine, tertiary amine, substituted amine including naturally occurring substituted amine, cyclic amine, and basic ion exchange resin such as isopropylamine, trimethylamine, diethylamine, triethylamine, tripropylamine, and ethanolamine. In certain embodiments, the pharmaceutically acceptable base addition salt can be selected from ammonium, potassium, sodium, calcium, and magnesium salts.

### (Additional Agents)

The combination of medical agents of the present disclosure may further comprise any additional agent. Additional agents include “chemotherapeutic agents”, which are useful compounds in the treatment of cancer, or “cytotoxic agents”, which are substances that inhibit or prevent cell function and/or cause cell death or destruction, but are not limited thereto. The additional agent may be formulated and administered as a separate composition from the other medical agents in the combination of medical agents of the present disclosure, or may be formulated and administered as a single composition with the other medical agents. When they are administered as separate compositions, they may be administered at a time or at different times (but within the period in which the effect of the rest of the medical agents on the subject persists). In addition, each medical agent can be administered in a different dosage form and/or method.

Cytotoxic agents include, but are not limited to: radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitors; enzymes and fragments thereof (e.g., nuclear degrading enzymes); and toxins (e.g., small molecule toxins of bacterial, fungal, plant or animal origin or toxins that are active as enzymes (including fragments and/or its variants)).

Exemplary cytotoxic agents can be selected from anti-microtubule agents, platinum coordination complexes, alkylating agents, antibacterial agents, topoisomerase II inhibitors, metabolic antagonists, topoisomerase inhibitors, hormone and hormone analogs, signal transduction pathway inhibitors, non-receptor tyrosine kinase angiogenesis inhibitors, immunotherapeutic agents, pro-apoptotic agents, LDH-A inhibitors; fatty acid biosynthesis inhibitors; cell cycle signaling inhibitors; HDAC inhibitors, proteasome inhibitors; and cancer metabolism inhibitors.

Examples of chemotherapeutic agents include: erlotinib (Tarceva®, Genentech/OSI Pharm.), bortezomib (Velcade®, Millennium Pharm.), disulfiram, epigallocatechin gallate, salinosporamide A, carfilzomib, 17-AAG (geldanamycin), radicicol, lactate dehydrogenase A (LDH-A), fulvestrant (Faslodex®, AstraZeneca), sunitinib (Sutent®, Pfizer/Sugen), letrozole (Femara®, Novartis), imatinib mesylate (Gleevec®, Novartis), finasunate (Vatalanib®, Novartis), oxaliplatin (Eloxatin®, Sanofi), 5-FU (5-fluorouracil), leucovorin, rapamycin (sirolimus, Rapamune®, Wyeth), lapatinib (Tykerb®, GSK572016, GlaxoSmithKline), lonafarnib (SCH 66336), sorafenib (Nexavar®, Bayer Labs), gefitinib (Iressa®, AstraZeneca), AG1478, alkylating agents (e.g., thiotepa and Cytoxan® cyclophosphamide); alkyl sulfonates (e.g., busulfan, improsulfan and piposulfan); aziridines (e.g., benzodopa, carboquone, methyldopa, and uredopa); ethyleneimines and methylmelamines (including altretamine, triethylenemelamine, triethylenephosphoramide, triethylenethiophosphoramide and trimethylomelamine); acetogenins (especially bullatacin and bullatacinone); camptothecin (including topotecan and irinotecan); bryostatin; callystatin; CC-1065 (including its synthetic analogs adozelesin, carzelesin and bizelesin); cryptophycin (particularly cryptophycin 1 and cryptophycin 8); adrenocorticosteroids (including prednisone and prednisolone); cyproterone acetate; 5α-reductase (including finasteride and dutasteride); vorinostat, romidepsin, panobinostat, valproic acid, mocetinostat dolastatin; aldesleukin, talc duocarmycin (including its synthetic analogs, KW-2189 and CB1-TM1); eleutherobin; pancratistatin; sarcodictyin; spongistatin; nitrogen mustard (e.g., chlorambucil, chlomaphazine, chlorophosphamide, estramustine, ifosfamide, mechlorethamine, mechlorethamine oxide hydrochloride, melphalan, novembichin, phenesterin, prednimustine, trophosfamide, uracil mustard); nitrosourea (e.g., carmustine, chlorozotocin, fotemustine, lomustine, nimustine, and ranimustine); antibiotics (e.g., enediyne antibiotics such as calicheamicin, especially calicheamicin γ1I and calicheamicin ω1I (Angew Chem. Intl. Ed. Engl. 1994 33: 183-186)); dynemicin (including dynemicin A); bisphosphonates (e.g., clodronate); esperamicin; and neocarzinostatin chromophores and related chromoprotein enediyne antibiotics), aclacinomycin, actinomycin, anthramycin, azaserine, bleomycin, cactinomycin, carubicin, caminomycin, carzinophilin, chromomycin, dactinomycin, daunorubicin, detorubicin, 6-diazo-5-oxo-L-norleucine, Adriamycin® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycin (e.g., mitomycin C, mycophenolic acid, nogalamycin, oligomycin, peplomycin, porfiromycin, puromycin, terramycin, rodorubicin, streptonigrin, streptozocin, tubercidin, ubenimex, zinostatin, zorubicin); metabolic antagonists (e.g., methotrexate and 5-fluorouracil (5-FU)); folic acid analogs (e.g., denopterin, methotrexate, pteropterin, trimetrexate); purine analogs (e.g., fludarabine, 6-mercaptopurine, thiamiprine, tioguanine); pyrimidine analogs (e.g., ancitabine, azacitidine, 6-azauridine, carmofur, cytarabine, dideoxyuridine, doxifluridine, enocitabine, floxuridine); androgens (e.g., calusterone, dromostanolone propionate, epitiostanol, mepitiostane, testolactone); anti-adrenals (e.g., aminoglutethimide, mitotane, trilostane); folic acid replenisher (e.g., folinic acid); aceglatone; aldophosphamide glycoside; aminolevulinic acid; eniluracil; amsacrine; bestrabucil; bisantrene; edatrexate; defofamine; demecolcine; diaziquone; eflornithine; elliptinium acetate; epothilone; ethoglucid; gallium nitrate; hydroxyurea; lentinan; lonidamine; maytansinoids (e.g., maytansine and ansamitocin); mitoguazon; mitoxantrone; mopidamol; nitraerine; pentostatin; phenamet; pirarubicin; losoxantrone; podophyllic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (particularly T-2 toxin, verrucarin A, roridin A and anguidin)); urethane; vindesin; dacarbazine; mannomustin; mitobronitol; mitolactol; pipobroman; gacytosine; Arabinoside (“Ara-C”); cyclophosphamide; thiotepa; taxoid (e.g., taxol (paclitaxel); Bristol-Myers Squibb Oncology, Princeton, N.J.), Abraxane® (Cremophor-free), albumin-bound nanoparticle formulation of paclitaxel (American Pharmaceutical Partners, Schaumberg, III.), and Taxotere® (docetaxel; Sanofi-Aventis); chlorambucil; Gemzar® (gemcitabine); 6-thioguanine; mercaptopurine; methotrexate; platinum analogs (e.g., cisplatin and carboplatin); vinblastine; etoposide (VP-16); ifosphamide; mitoxantrone; vincristine; navelbine® (vinorelbine); novantron; teniposide; edatrexate; daunomycin; aminopterin; capecitabine (Xeloda®); ibandronate; CPT-11; topoisomerase inhibitor RFS 2000; difluoromethylornithine (DMFO); retinoids (e.g., retinoic acid); and pharmaceutically acceptable salts, acids and derivatives of any of the above.

Chemotherapeutic agents also include: (i) antihormonal agents that act to regulate or inhibit hormonal action on tumors (e.g., anti-estrogen and selective estrogen receptor modulators (SERMs) (e.g., tamoxifen (Nolvadex® including tamoxifen citrate), raloxifene, droloxifene, endoxifen, 4-hydroxytamoxifen, trioxifen, keoxifene, LY117018, onapristone, and Fareston® (toremifene citrate))); (ii) aromatase inhibitors that inhibit the enzyme aromatase that regulates estrogen production in the adrenal cortex (e.g., 4(5)-imidazole, aminoglutetimide, Megace® (megestrol acetate), Aromasin® (exemestane; Pfizer), formestane, fadrozole, Rivizor® (vorozole), Femara® (letrozole; Novartis), and Arimidex® (anastrozole; Astra Zeneca)); (iii) anti-androgens (e.g., flutamide, nilutamide, bicalutamide, leuprolide and goserelin; buserelin, triptorelin, medroxyprogesterone acetate, diethylstilbestrol, premarin, fluoxymesterone, all-trans-retinoic acid, fenretinide, and troxacitabine (1,3-dioxolane nucleoside cytosine analog); (iv) protein kinase inhibitors; (v) lipid kinase inhibitors; (vi) antisense oligonucleotides (particularly those that inhibit expression of genes in the signaling pathway involved in ectopic cell proliferation, such as PKC-α, Ralf and H-Ras); (vii) ribozymes (e.g., VEGF expression inhibitors (e.g., Angiozyme®) and HER2 expression inhibitors); (viii) vaccines (e.g., gene therapy vaccines such as Allovectin®, Leuvectin®, and Vaxid®); Proleukin®, rIL-2; topoisomerase 1 inhibitors (e.g., Lurtotecan®; Abarelix® rmRH); and (ix) pharmaceutically acceptable salts, acids and derivatives of any of the above.

Chemotherapeutic agents also include: antibodies (e.g., alemtuzumab (Campath), bevacizumab (Avastin®, Genentech); cetuximab (Erbitux®, Imclone); panitumumab (Vectibix®), Amgen), rituximab (Rituxan®, Genentech/Biogen Idec), pertuzumab (Omnitag®, 2C4, Genentech), trastuzumab (Herceptin®, Genentech), tositumomab (Bexxar, Corixia), and antibody-drug conjugates, gemtuzumab ozogamicin (Mylotarg®, Wyeth). Further humanized monoclonal antibodies that have therapeutic potential in combination with the compound of the present disclosure include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motobizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, tralizumab, tucotuzumab celmoleukin, tucusituzumab, omalizumab, urtoxazumab, ustekinumab, visilizumab, and anti-interleukin-12 antibodies that exclusively comprise human recombinant sequences (ABT-874/J695, Wyeth Research and Abbott Laboratories), a full-length IgG₁ λ antibody genetically modified to recognize interleukin-12 p40 protein.

Chemotherapeutic agents also include “EGFR inhibitors”, which refer to compounds that bind to or otherwise interact directly with EGFR and prevent or reduce its signaling activity and also are referred to as “EGFR antagonists”. Examples of such agents include antibodies and small molecules that bind to EGFR. Examples of antibodies that bind to EGFR include: MAb 579 (ATCC CRL HB 8506), MAb 455 (ATCC CRL HB8507), MAb225 (ATCC CRL 8508), MAb 528 (ATCC CRL 8509) (see U.S. Pat. No. 4,943,533, Mendelsohn et al.) and their variants (e.g., a chimeric 225 (C225 or cetuximab; ERBUTIX®) and a reshaped human 225 (H225) (see WO96/40210, Imclone Systems Inc.)); IMC-11F8, a fully human EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR (e.g., ABX-EGF or panitumumab (see WO98/50433, Abgenix/Amgen)); EMD 55900 (Stragliotto et al., Eur. J. Cancer 32A: 636-640 (1996)); EMD7200 (matuzumab), a humanized EGFR antibody directed against EGFR that competes with both EGF and TGF-α for EGFR binding (EMD/Merck); a human EGFR antibody, HuMax-EGFR (GenMab); fully human antibodies known as E1.1, E2.4, E2.5, E6.2, E6.4, E2.11, E6.3 and E7.6.3, and described in U.S. Pat. No. 6,235,883; MDX-447 (Medarex Inc); and mAb 806 or humanized mAb 806 (Johns et al., J. Biol. Chem. 279(29): 30375-30384 (2004)). The anti-EGFR antibody can be conjugated to a cytotoxic agent and thus can generate an immunoconjugate (see, e.g., EP659,439A2, Merck Patent GmbH). Examples of EGFR antagonists include small molecules such as compounds described in the following US patents: U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, [symbol] 5,747,498, and the following PCT publications: WO98/14451, WO98/50038, WO99/09016, and WO99/24037. Specific small molecule EGFR antagonists include: OSI-774 (CP-358774, erlotinib (Tarceva® Genentech/OSI Pharmaceuticals); PD 183805 (CI 1033, 2-propenamide, N-[4)-[(3-chloro-4-fluorophenyl)amino]-7-[3-(4-morpholinyl)propoxy]-6-quinazolinyl]-, dihydrochloride, Pfizer Inc.); ZD1839, gefitinib (Iressa®))) 4-(3′-chloro-4′-fluoroanilino-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); double EGFR/HER2 tyrosine kinase inhibitor (e.g., lapatinib (Tykerb®), GSK572016 or N-[3-chloro-4-[(3-fluorophenyl)methoxy]phenyl]-6-[5-[[[2-methylsulfonyl)ethyl]amino]methyl]-2-furanyl]-4-quinazoline amine)).

Chemotherapeutic agents also include: “tyrosine kinase inhibitors” (including EGFR-targeted agents); small molecule HER2 tyrosine kinase inhibitors (e.g., TAK165 available from Takeda); CP-724,714, an oral selective inhibitor of ErbB2 receptor tyrosine kinase (Pfizer and OSI); dual HER inhibitors that preferentially bind to EGFR but inhibit both HER2 overexpressing cells and EGFR overexpressing cells (e.g., EKB-569 (available from Wyeth)); lapatinib (GSK572016; available from Glaxo-SmithKline), oral HER2 and EGER tyrosine kinase inhibitors; PKI-166 (available from Novartis); pan-HER inhibitors (e.g., canertinib (CI-1033; Pharmacia)); Raf-1 inhibitors (e.g., ISIS-5132 available from ISIS Pharmaceuticals, which is an antisense agent that inhibits Raf-1 signaling); non-HER-targeted TK inhibitors (e.g., imatinib mesylate (Gleevec®, available from Glaxo SmithKline); multi-targeted tyrosine kinase inhibitors (e.g., sunitinib (Sutent®, available from Pfizer)); VEGF receptor tyrosine kinase inhibitors (e.g., vatalanib (PTK787/ZK222584, available from Novartis/Schering AG); a MAPK extracellular regulatory kinase I inhibitor CI-1040 (available from Pharmacia); quinazolines (e.g., PD 153035, 4-(3-chloroanilino)quinazoline); pyridopyrimidines; pyrimidopyrimidines; pyrrolopyrimidines (e.g., CGP 59326, CGP 60261 and CGP 62706); pyrazolopyrimidines, 4-(phenylamino)-7H-pyrrilo[2,3-d]pyrimidine; curcumin (diferuloyl methane), 4,5-bis(4-fluoroanilino)phthalimide); tyrphostin containing a nitrothiophene moiety; PD-0183805 (Warner-Lamber); antisense molecules (e.g., those that bind to HER-coding nucleic acids); quinoxaline (U.S. Pat. No. 5,804,396); tyrphostin (U.S. Pat. No. 5,804,396); ZD6474 (Astra Zeneca); PTK-787 (Novartis/Schering AG); pan-HER inhibitors (e.g., CI-1033 (Pfizer)); affinitak (ISIS 3521; Isis/Lilly); imatinib mesylate (Gleevec®); PKI 166 (Novartis); GW2016 (Glaxo Smith Kline); CI-1033 (Pfizer); EKB-569 (Wyeth); semaxanib (Pfizer); ZD6474 (AstraZeneca); PTK-787 (Novartis/Schering AG); INC-1C11 (Imclone), rapamycin (sirolimus, RapaMune®); or those described in any of the following patent publications: U.S. Pat. No. 5,804,396; WO1999/09016 (American Cyanamid); WO1998/43960 (American Cyanamid); WO1997/38983 (Warner Lambert); WO1999/06378 (Warner Lambert); WO1999/06396 (Warner Lambert); WO1996/30347 (Pfizer, Inc); WO1996/33978 (Zeneca); WO1996/3397 (Zeneca) and WO1996/33980 (Zeneca).

Chemotherapeutic agents also include: dexamethasone, interferon, colchicine, metoprin, cyclosporin, amphotericin, metronidazole, alemtuzumab, alitretinoin, allopurinol, amifostine, arsenic trioxide, asparaginase, BCG raw, bevacizumab, bexarotene, cladribine, clofarabine, darbepoetin α, denileukin, dexrazoxane, epoetin α, erlotinib, filgrastim, histrelin acetate, ibritumomab, interferon α-2a, interferon α-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include: hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone 17-butyrate, hydrocortisone 17-valerate, alclometasone dipropionate, betamethasone valerate, betamethasone dipropionate, prednicarbate, clobetasone 17-butyrate, clobetasol 17-propionate, fluocortolone caproate, fluocortolone pivalate and fluprednidene acetate; immunoselective anti-inflammatory peptides (ImSAIDs) (e.g., phenylalanine-glutamine-glycine (FEG) and its D-isomer form (feG) (IMULAN BioTherapeutics, LLC)); antirheumatic agents (e.g., azathioprine, cyclosporin (ciclosporin) (cyclosporin A), D-penicillamine, gold salts, hydroxychloroquine, leflunomide, minocycline, sulfasalazine), tumor necrosis factor α (TNFα) blockers (e.g., etanercept (Enbrel), infliximab (Remicade), adalimumab (Humira), certolizumab pegol (Cimzia), golimumab (Simponi), interleukin 1 (IL-1) blockers (e.g., Anakinra (Kineret)), T cell costimulatory blockers (e.g., abatacept (Orencia)), interleukin 6 (IL-6) blockers (e.g., tocilizumab (ACTEMERA®)); interleukin 13 (IL-13) blockers (e.g., lebrikizumab); interferon α (IFN) blockers (e.g., rontalizumab); B7 integrin blockers (e.g., rhuMAb β7); TgE pathway blockers (e.g., anti-M1 prim); secreted homotrimer LTa3 and membrane-bound heterotrimmer LTa1/β2 blockers (e.g., anti-lymphotoxin α (LTa)); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); various investigational agents (e.g., thioplatin, PS-341, phenylbutyrate, ET-18-OCH₃, or farnesyltransferase inhibitors (L-739749, L-744832)); polyphenols (e.g., quercetin, resveratrol, piceatannol, epigallocatechin gallate, theaflavin, flavanol, procyanidin, betulinic acid and derivatives thereof); autophagy inhibitors (e.g., chloroquine); δ-9-tetrahydrocannabinol (dronabinol, Marinol®); β-lapachone; lapachol; colchicine; betulinic acid; acetyl camptothecin, scopoletin, and 9-aminocamptothecin); podophyllotoxin; Tegafur (UFTORAL®); bexarotene (Targretin®); bisphosphonates (e.g., clodronate (e.g., Bonefos® or Ostac®), etidronate (DIDROCAL®), NE-58095, zoledronic acid/zoledronate (Zometa®)), alendronate (Fosamax®), pamidronate (Aredia®), tiludronate (Skelid®), or risedronate (Actonel®)); and epidermal growth factor receptor (EGF-R); vaccines (e.g., Theratope® vaccine); perifosine, COX-2 inhibitors (e.g., celecoxib or etoricoxib), proteasome inhibitors (e.g., PS341); CCI-779; tipifarnib (R11577); orafenib, ABT510; Bcl-2 inhibitors (e.g., oblimersen sodium (Genasense®)); pixantrone; farnesyltransferase inhibitors (e.g., lonafarnib (SCH 6636, Salazar™)); and any pharmaceutically acceptable salts, acids or derivatives of any of the above; and combinations of two or more of the above (e.g., CHOP (abbreviation for combination therapy with cyclophosphamide, doxorubicin, vincristine, and prednisolone); and FOLFOX (abbreviation for treatment regimen with oxaliplatin (eloxatin™) in combination with 5-FU and leucovorin).

Chemotherapeutic agents also include non-steroidal anti-inflammatory agents with analgesic, antipyretic and anti-inflammatory effects. NSAIDs include non-selective inhibitors of the enzyme cyclooxygenase. Specific examples of NSAIDs include: aspirin, propionic acid derivatives (e.g., ibuprofen, fenoprofen, ketoprofen, flurbiprofen, oxaprozin and naproxen), acetic acid derivatives (e.g., indomethacin, sulindac, etodolac, diclofenac), enolic acid derivatives (e.g., piroxicam, meloxicam, tenoxicam, droxicam, lornoxicam and isoxicam), fenamic acid derivatives (e.g., mefenamic acid, meclofenamic acid, flufenamic acid, tolfenamic acid), and COX-2 inhibitors (e.g., celecoxib, etoricoxib, lumiracoxib, parecoxib, rofecoxib, rofecoxib, and valdecoxib). NSAIDs can be indicated for relief of conditions such as rheumatoid arthritis, osteoarthritis, inflammatory arthritis, ankylosing spondylitis, psoriatic arthritis, Reiter's syndrome, acute gout, dysmenorrhea, metastatic bone pain, headache and migraine, postoperative pain, mild to moderate pain resulting from inflammation and tissue damage, fever, ileus, and renal colic.

Chemotherapeutic agents may also include: therapeutic agents for Alzheimer's disease (e.g., donepezil hydrochloride and rivastigmine); treatments for Parkinson's disease (e.g., L-dopa/carbidopa, entacapone, ropinirole, pramipexole, bromocriptine, pergolide, trihexyphenidyl, and amantadine; agents for treating multiple sclerosis (MS) (e.g., β-interferon (e.g., Avonex® and Rebif®), glatiramer acetate, and mitoxantrone); treatments for asthma (e.g., albuterol and montelukast sodium); agents for treating schizophrenia (e.g., Zyprexa, Risperdal, Seroquel, and haloperidol); anti-inflammatory agents (e.g., corticosteroids, TNF blockers, IL-1 RA, azathioprine, cyclophosphamide, and sulfasalazine; immunomodulatory and immunosuppressive agents (e.g., cyclosporin, tacrolimus, rapamycin, mycophenolate mofetil, interferons, corticosteroids, cyclophosphamide, azathioprine, and sulfasalazine; neurotrophic factors (e.g., acetylcholinesterase inhibitors, MAO inhibitors, interferons, antiepileptic agents, ion channel blockers, riluzole, and antiparkinson agents); agents for treating cardiovascular diseases (e.g., β-blockers, ACE inhibitors, diuretics, nitrates, calcium channel blockers, and statins); agents for treating liver diseases (e.g., corticosteroids, cholestyramine, interferons, and antiviral agents); agents for treating blood disorders (e.g., corticosteroids, antileukemic agents and growth factors); and agents for treating immunodeficiency disorders (e.g., γ-globulin).

In addition, chemotherapeutic agents include pharmaceutically acceptable salts, acids or derivatives of any of the chemotherapeutic agents described herein, as well as combinations of two or more of them.

### (Embodiments of Combination)

A pharmaceutical composition for the treatment of unresectable advanced/recurrent colorectal cancer can be provided. A pharmaceutical composition for the treatment of unresectable advanced/recurrent colorectal cancer in PD-L1 positive patients can be provided. A pharmaceutical composition for the treatment of unresectable advanced/recurrent right-sided colorectal cancer in PD-L1 positive patients can be provided. A pharmaceutical composition for the treatment of unresectable advanced/recurrent right-sided colorectal cancer (microsatellite stable) in PD-L1 positive patients can be provided. The pharmaceutical composition can comprise napabucasin as an active ingredient. The pharmaceutical composition, in combination with pembrolizumab, can usually be administered daily to adults at 240 mg/day as napabucasin. For testing, approved extracorporeal diagnostic agents can be used. For testing PD-L1 positive expression, PD-L1 IHC 22C3 pharmDx “Dako” can be used. The microsatellite stability test can be a microsatellite instability (MSI) test by PCR or a mismatch repair (MMR)-related protein test by immunochemical staining (IHC).

A pharmaceutical composition for the treatment of unresectable advanced/recurrent colorectal cancer classified as CMS1 or CMS4 can be provided. A pharmaceutical composition for the treatment of unresectable advanced/recurrent right-sided colorectal cancer classified as CMS1 or CMS4 can be provided. A pharmaceutical composition for the treatment of unresectable advanced/recurrent colorectal cancer (microsatellite stable) classified as CMS1 or CMS4 can be provided. A pharmaceutical composition for the treatment of unresectable advanced/recurrent right-sided colorectal cancer (microsatellite stable) classified as CMS1 or CMS4 can be provided. The pharmaceutical composition can comprise napabucasin as an active ingredient. The pharmaceutical composition, in combination with pembrolizumab, can usually be administered daily to adults at 240 mg/day as napabucasin. CMS classification can be determined by the subtype classification algorithm of Guinney et al. (Guinney J, et al.: Nat Med. 21(11):1350-1356, 2015) by measuring gene expression in tumor tissues by RNA sequencing or microarray, for example.

In one embodiment, in the pharmaceutical composition or therapeutic method provided by the present disclosure, the patient as mentioned above is identified as a subject to be treated, and these features may be described in a package insert or label to give instructions to physicians and other healthcare professionals involved in the treatment It is also possible to give treatment guidelines to physicians or others from other information sources than these official documents.

### EXAMPLES

### Example 1

### Efficacy of Combined Agents in MSS Patients

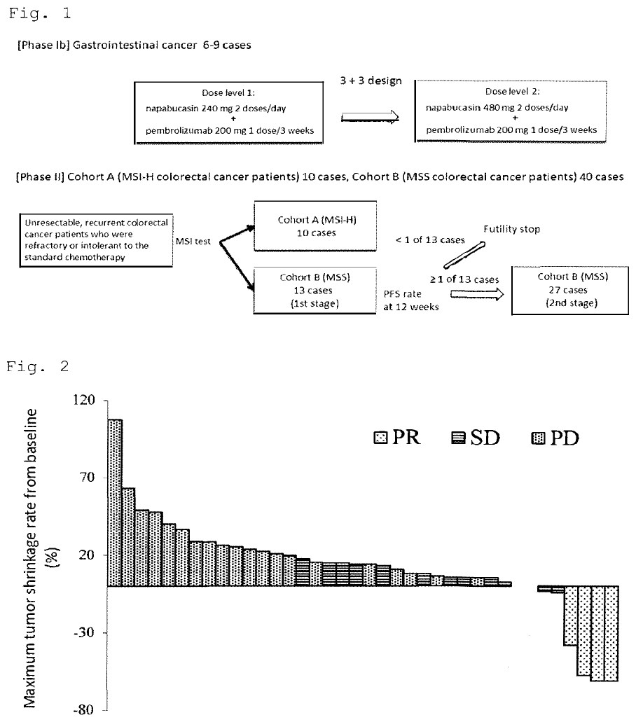

The outline of the test design in this example is shown in FIG. 1. This was an open-label, multicenter, phase Ib/II clinical trial, and an exploratory evaluation of effectiveness and safety of combination use of napabucasin and pembrolizumab in patients with unresectable, recurrent colorectal cancer who were refractory or intolerant to the standard chemotherapy. In phase Ib, the safety of combination use of napabucasin and pembrolizumab was evaluated in patients with unresectable, recurrent gastrointestinal cancer who were refractory or intolerant to the standard chemotherapy, and the recommended dose of napabucasin in this combination use was determined. In phase II, the effectiveness and safety of combination use of napabucasin and pembrolizumab were exploratorily evaluated in patients with unresectable, recurrent colorectal cancer who were refractory or intolerant to the standard chemotherapy in cohort A (microsatellite instable colorectal cancer) and in cohort B (microsatellite stable colorectal cancer). The primary endpoint was objective response rate by ir RECIST, and the secondary endpoints were progression-free survival at 12 weeks by ir RECIST (irPFS: immune-related progression free survival), objective response rate (ORR) by RECIST version 1.1, progression free survival (PFS) at 12 weeks by RECIST version 1.1, progression free survival (PFS), overall survival (OS), disease control rate (DCR), incidence of adverse events and pharmacokinetic parameters. In addition, as an exploratory item, correlation between PD-L1 expression and efficacy and safety was examined. The subjects in the target group in this study were those who met the following conditions.
1. Patients who gave a written consent to be subjected to this clinical trial.
2. Patients who were 20 years or older on the day they gave the consent.
3. In phase Ib, patients with histologically confirmed gastrointestinal cancer, and in phase II, patients with histologically confirmed adenocarcinoma in colon or rectal cancer, confirmed presence or absence of mutations of at least codons 12 and 13 of KRAS gene by RAS genetic testing, and confirmed MSI status.
4. Regarding chemotherapy history, in phase Ib, patients with gastrointestinal cancer who were refractory or intolerant to the standard chemotherapy, and in phase II, patients who received one or more regimens of the following standard chemotherapies for metastatic colorectal cancer but were refractory or intolerant to the chemotherapies.
5. Patients with ECOG Performance Status (P.S.) of 0 or 1,
6. Patients who were able to receive oral administration of agents.
7. Patients with evaluable lesions (phase Ib and cohort A in phase II) or measurable lesions (cohort B in phase II) as specified in RECIST version 1.1.
8. Patients with sufficient organ function.
9. Women who were negative on pregnancy test within 7 days prior to enrollment when they could get pregnant.

Patients who agreed to use appropriate contraception during the study and up to 4 months after discontinuation of the study agents were selected for both men and women.

The subjects repeatedly received administration of the study agents until they got to meet the discontinuation criteria. In phase Ib, one course for administration of the study agents consisted of 21 days, and 240 mg or 480 mg napabucasin was taken twice daily, and 200 mg/body pembrolizumab was administered on day 1 of each cycle.

In phase II, one course consisted of 21 days, and 480 mg napabucasin was orally administered twice daily and pembrolizumab was administered on day 1 of each cycle. A total of 8 subjects were evaluated in phase Ib, and in phase II, 10 subjects were evaluated in cohort A and 40 subjects in cohort B.

### Results

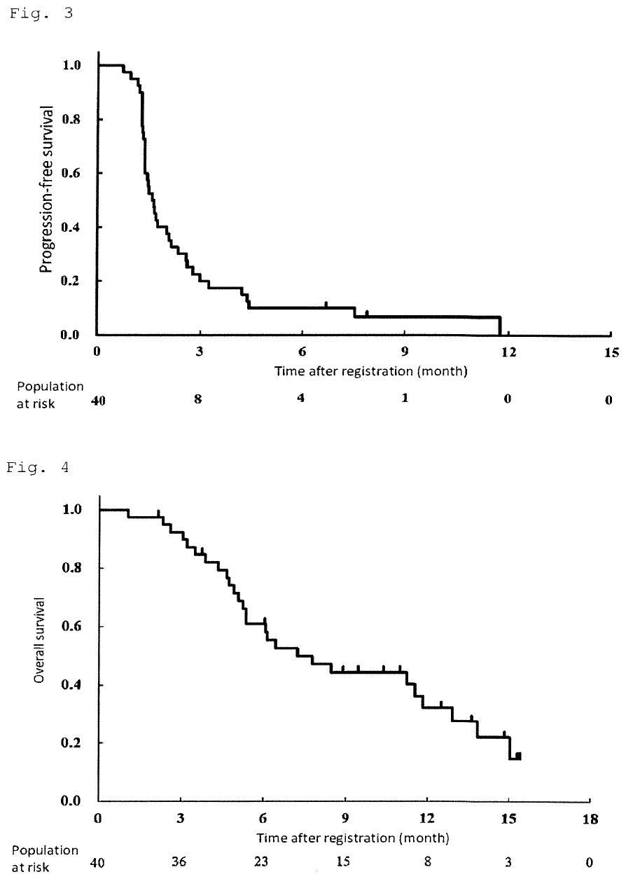

Results are shown in FIGS. 2 to 6.

Tumor shrinkage effect of the napabucasin and pembrolizumab combination therapy for MSS colorectal cancer patients is shown in the Waterfall plot in FIG. 2. For the subjects enrolled in this study, chest, abdomen, and pelvic CT/MRI examinations were performed before the start of treatment and at 6, 12, 18, and 24 weeks after the start of treatment (and then every 9 weeks until the 42nd week, and every 12 weeks after the 42nd week) to evaluate the tumor shrinkage effect of the combination therapy. The tumor shrinkage effect was determined by new response evaluation criteria in solid tumors (RECIST guideline) ver1.1 or irRECIST (immune-related response criteria), which applied the RECIST v1.1 to the evaluation method of tumor shrinkage effect seen in immunotherapy. As a result, PR (Partial Response: defined as at least a 30% decrease in the sum of diameters of the target lesion, taking as reference the baseline sum diameter) was observed in 4 subjects with MSS colorectal cancer as the best overall response. The objective response rate (ORR) was 10%.

The progression free survival (PFS) in MSS colorectal cancer patients is shown in FIG. 3. Of all the enrolled subjects, progression free survival (PFS) was evaluated in patients who received the dose recommended for the phase II part and had measurable lesions (FAS: Full Analysis Set). The OS was defined as the period from the date of registration to the date of exacerbation or the date of death due to any cause, whichever came first. The survival function for the PFS was estimated using the Kaplan-Meier method. The median PFS for MSS colorectal cancer was 1.6 months.

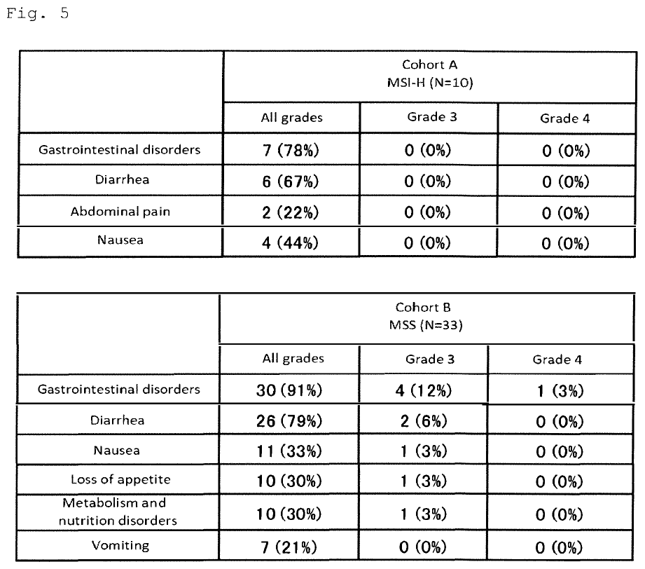

The overall survival (OS) in MSS colorectal cancer patients is shown in FIG. 4. The overall survival (OS) was evaluated in FAS. The PFS was defined as the period from the date of registration to the date of death due to any cause. The OS was also analyzed in the same way as the PFS. As a result, the median OS for MSS colorectal cancer was 7.3 months.

In multiple previous studies, a PD-(L)1 inhibitor was administered to more than 150 MSS (non-MSI-H) colorectal cancer patients, but response (ORR) was observed only in three cases. Of these three responses, one nivolumab-administered case and one atezolizumab-administered case were found to be actually MSI-H, and the MSS/MSI status of the remaining one was unknown.

TABLE 1
Treatment results of PD-(L) 1 inhibitors

| | | | | Number of | | |
|---|---|---|---|---|---|---|
| | | Type of | | patients | | ClinicalTrials.gov |
| Authors | Issue | cancer | Agent | (N) | ORR | ID |
| Brahmer | 2010 | CRC | nivolumab | 14 | 7 | |
| | | | | | (1/14) | |
| Topialan | 2012 | Refractory CRC | nivolumab | 19 | 0 | NCT00730639 |
| et al. | | | | | | |
| Le | 2015 | MSS CRC | pembrolizumab | 18 | 0 | NCT01876511 |
| et al. | | | | | | |
| O' Neil | 2017 | PD-1 positive | pembrolizumab | 22 | 0 | NCT02054806 |
| et al. | | MSS CRC | | | | |
| Bendell | 2018 | MSS CRC | atezolizumab | 90 | 2.2% | NCT02788279 |
| et al. | | | | | (2/90) | |

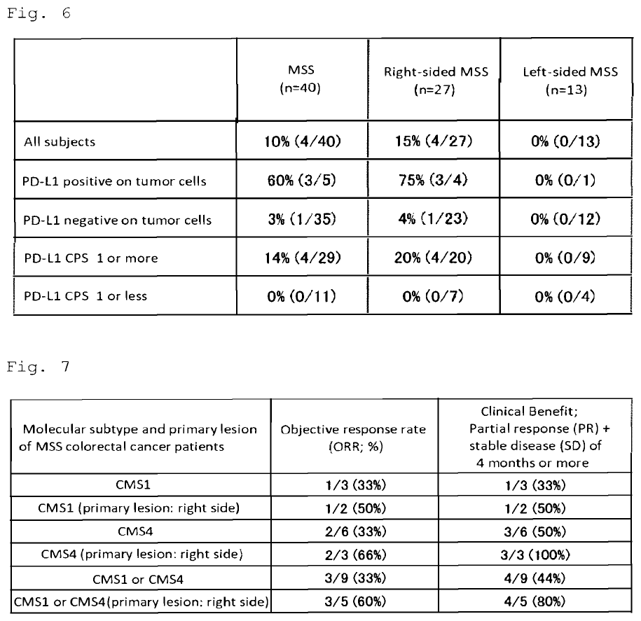

FIG. 5 shows the frequency and grade of adverse events that occurred in subjects who received the napabucasin and pembrolizumab combination therapy. Adverse events (subjective and objective symptoms) were observed and recorded from the start of treatment to 30 days after the final administration of the protocol treatment. The adverse event was defined as any unfavorable or unintended sign (including abnormal laboratory test value), symptom or disease that occurred in subjects who received the study agents regardless of whether or not it had a causal relationship with the study agents. As the incidence of adverse event, the frequency of the worst grade according to CTCAE v4.03-JCOG in the whole course was calculated for each adverse event for the population who received this study treatment at least once (SP: Safety Population) among all registered patients. Adverse events that occurred in more than 20% of SP in cohort A and cohort B were diarrhea, vomiting, loss of appetite, and vomiting. The frequency and severity of adverse events were not significantly different from those with monotherapy of napabucasin or pembrolizumab, and this combination therapy was tolerable.

The ORR in the presence or absence of PD-L1 expression is shown in FIG. 6. From 40 MSS colorectal cancer subjects and 10 MSI-High colorectal cancer subjects who could submitted tumor specimens at baseline, sections having a thickness of 5 μm were cut from formalin-fixed paraffin-embedded (FFPE) tissue blocks and stained with PD-L1 IHC 22C3 pharmDx “Dako”. Of 5 MSS colorectal cancer subjects whose PD-L1 expression was 1% or higher on tumor cells, 4 had right-sided colorectal cancer and 1 had left-sided colorectal cancer. The ORRs of MSS right-sided and left-sided colorectal cancers that were positive for PD-L1 on tumor cells were 3/4 (75%) and 0/1 (0%), respectively. Of 29 MSS colorectal cancer subjects whose Combined Positive Score (CPS) (combined PD-L1 expression on tumor cells and immune cells) was 1% or more, 20 had right-sided colorectal cancer and 9 had left-sided colorectal cancer. The ORRs of MSS right-sided and left-sided colorectal cancers with CPS of 1% or more were 4/20 (20%) and 0/9 (0%), respectively. These results suggest that the characteristics “right-sided” and “PD-L1 positive” in MSS colorectal cancer patients can be useful factors to predict the therapeutic effect of this combination therapy.

The ORR or Clinical Benefit (defined as “PR+SD>4 months”) for colorectal cancer patients classified as CMS1 or CMS4 is shown in FIG. 7. From subjects who could submitted tumor specimens at baseline, RNA was extracted from tumor tissues and gene expression was analyzed by RNA sequencing, and the subjects were classified into CMS1 to CMS4 according to the molecular subtype based on a classification method proposed by the Colorectal Cancer Subtyping Consortium (CRCSC) (Guinney J, et al.: Nat Med. 21(11): 1350-1356, 2015). Of 31 subjects that could submit tumor specimens at baseline, 5 were CMS1 (including 2 MSI colorectal cancer), 6 were CMS2, 4 were CMS3, 6 were CMS4, and 10 were unknown or unmeasurable. The ORR and Clinical Benefit of MSS colorectal cancer patients classified as CMS1 or CMS4 were 3/9 cases (33%) and 4/9 cases (44%), respectively, and the ORR and Clinical Benefit of MSS colorectal cancer patients classified as CMS1 or CMS4 whose primary lesion was on the right side was 3/5 cases (60%) and 4/5 cases (80%), respectively.

On the other hand, the ORR of MSS colorectal cancer patients classified as CMS2 and CMS3 were 0/6 cases (0%) and 1/4 cases (25%), respectively. One successful case with CMS3 had a mutation in the polymerase ε (POLE), a DNA polymerase involved in DNA replication and repair. Similar to MSI, POLE mutations indicate phenotypes with high mutation load in tumors and are therefore considered as promising predictive markers for immune checkpoint inhibitors, and successful cases of pembrolizumab monotherapy have been reported in colorectal cancer patients with POLE genetic mutations (Laetitia Nebot-Bral, et al. European Journal of Cancer 84 (2017) 290e303, J. Gong, et al, J. Natl. Compr. Canc. Netw. (2) (2017) 142-147). From this, it was speculated that one responsive patient with CMS3 might also respond to pembrolizumab monotherapy. These results suggest that CMS1 or CMS4 can be a useful factor to predict the therapeutic effect of this combination therapy in patients with MSS colorectal cancer.

### Example 2

### AI/IoT/Cloud Service (SaaS)

Provided is a system comprising a storage for storing patient characteristic information of a plurality of patients and responsiveness information of the plurality of individuals, a learning unit configured to learn relationship between a patient characteristic and responsiveness from the patient characteristic information and the responsiveness information of the plurality of individuals, a processor that predicts responsiveness of a patient from a patient characteristic of the patient based on the relationship between the patient characteristic and responsiveness.

Information such as microsatellite stability information, PD-L1 expression information, cancer primary site information or CMS information as well as information on responsiveness to a cancer treatment are obtained from a plurality of patients and stored in the storage. The obtained information is served for machine learning and a discriminant model is generated.

From a patient whose responsiveness is to be predicted, information such as microsatellite stability information, PD-L1 expression information, cancer primary site information or CMS information is obtained. The obtained information is applied to the discriminant model in the processor to predict responsiveness to the cancer treatment. The information may be transmitted from a terminal installed in a hospital or other places to a processor on the cloud service that stores the discriminant model. The result is returned to the terminal, or a display is used to transmit the result to the user.

### (Note)

As described above, the present disclosure has been illustrated using the preferred embodiments of the present disclosure, but the present disclosure should not be construed as being limited to these embodiments. It is understood that the present disclosure should be construed only by the claims. It will be understood from those skilled in the art that the description of the specific preferred embodiments of the present disclosure will enable equivalent scope to be implemented based on the description of the present disclosure and common general technical knowledge. It is understood that the patents, patent applications and documents cited herein should be herein incorporated by reference in their entities as if they are specifically described herein as reference to this specification. This application claims priority to Japanese Patent Application No. 2019-153109 filed with the Japan Patent Office on Aug. 23, 2019, and it is understood that the content of the application should be herein incorporated by reference in its entity as if it is specifically described herein as reference to this specification.

### INDUSTRIAL APPLICABILITY

The present disclosure is useful for cancer therapeutic agents or companion diagnostic agents for cancer therapeutic agents, or any procedures in which they are used.

### DESCRIPTION OF CODE

101: System
102: Storage
103: Learning unit
104: Processer
105: Display
106: Database
107: Measurement unit

1-48. (canceled)

49. A method for treating a microsatellite stable (MSS) cancer patient, comprising administering to the patient an effective amount of a cancer stem cell inhibitor and an effective amount of an immune checkpoint inhibitor, wherein the patient has one or more patient characteristics that indicate that the patient is responsive to the cancer treatment.

50. The method according to claim 49, wherein the cancer stem cell inhibitor is administered separately from the immune checkpoint inhibitor.

51. The method according to claim 49, wherein the cancer stem cell inhibitor is administered together with the immune checkpoint inhibitor.

52. The method according to claim 49, wherein the one or more patient characteristics comprise a characteristic that the cancer is colorectal cancer.

53. The method according to claim 52, wherein the one or more patient characteristics comprise a characteristic that the cancer is right-sided colorectal cancer.

54. The method according to claim 49, wherein the one or more patient characteristics comprise a characteristic that PD-L1 expression is positive on immune cells of the patient.

55. The method according to claim 49, wherein the one or more patient characteristics comprise a characteristic that PD-L1 expression is positive on tumor cells of the patient.

56. The method according to claim 49, wherein the one or more patient characteristics comprise a characteristic that CMS of the patient is 1 or 4.

57. The method according to claim 49, wherein the one or more patient characteristics comprise characteristics
(1) the cancer is right-sided colorectal cancer, and PD-L1 expression is positive on immune cells of the patient,
(2) the cancer is right-sided colorectal cancer, and PD-L1 expression is positive on tumor cells of the patient, or
(3) the cancer is right-sided colorectal cancer, and CMS of the patient is 1 or 4.

58. The method according to claim 49, wherein the immune checkpoint inhibitor is an inhibitor of PD-L1 or PD-1

59. The method according to claim 49, wherein the immune checkpoint inhibitor is an anti-PD-1 antibody.

60. The method according to claim 49, wherein the cancer stem cell inhibitor is a STATS pathway inhibitor.

61. The method according to claim 49, wherein the cancer stem cell inhibitor is
a compound of Formula I : 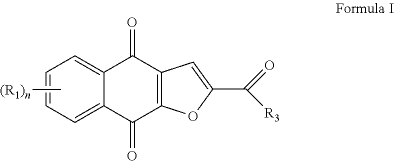
wherein,
each (R₁) is independently selected from the group consisting of hydrogen, halogen, fluorine, cyano, nitro, CF₃, OCF₃, alkyl, methyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NH₂,
n is 0, 1, 2, 3, or 4,
R₃ is selected from the group consisting of hydrogen, halogen, fluorine, cyano, CF₃, OCF₃, alkyl, methyl, substituted alkyl, halogen-substituted alkyl, hydroxyl-substituted alkyl, amine-substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, substituted aryl, ORₐ, SRₐ, and NR_{b}R_{c}, wherein Rₐ is independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, heterocycle, substituted heterocycle, aryl, and substituted aryl, and R_{b} and R_{c} are independently selected from the group consisting of hydrogen, alkyl, substituted alkyl, cycloalkyl, substituted cycloalkyl, heterocycle, substituted heterocycle, aryl, and substituted aryl, or R_{b} and R_{c} form a heterocycle or a substituted heterocycle together with the N to which they are attached, or
a compound of Formula 1A: 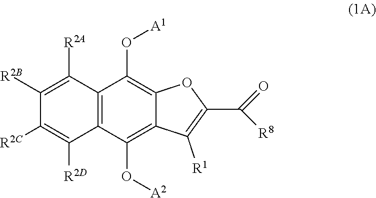
wherein A¹ and A² are the same or different and independently —C(═O)B, —CO₂B, —CONR^{3C}B, or a hydrogen atom, wherein A¹ and A² are not hydrogen atoms at the same time,
wherein B is
(1) a 3- to 6-membered monocyclic or polycyclic heterocyclic group,
(2) a 3- to 6-membered cyclic amino group, or
(3) a group represented by Formula (B): 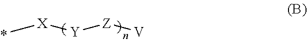
wherein the 3- to 6-membered monocyclic heterocyclic group and the 3- to 6-membered cyclic amino group have at least one secondary nitrogen atom in the ring, and
in Formula (B),
X is
(1) a single bond,
(2) C₁₋₆ alkylene, wherein the alkylene is optionally substituted with 1 to 3 substituents selected from the group consisting of a carboxyl group and —CO₂R⁶, or
(3) C₃₋₁₀ cycloalkylene,
Y is a single bond, an oxygen atom, or —NR^{4A}—,
R^{4A} is a hydrogen atom,
Z is
(1) a single bond or
(2) C₁₋₆ alkylene,
n is 0 or 1,
V is
(1) —NHR⁵,
(2) a 3- to 6-membered monocyclic or polycyclic heterocyclic group, or
(3) a 3- to 6-membered cyclic amino group,
wherein the 3- to 6-membered monocyclic or polycyclic heterocyclic group and the 3- to 6-membered cyclic amino group have at least one secondary nitrogen atom in the ring,
R⁵ is
(1) a hydrogen atom or
(2) a C₁₋₆ alkyl group, wherein the alkyl group is optionally substituted with 1 to 3 substituents selected from the group consisting of a halogen atom, a hydroxyl group, a carboxyl group, a sulfinic acid group, a sulfonic acid group, a phosphoric acid group, a C₆-₁₀ aryl group, a C₁₋₆ alkoxy group, a C₃₋₈ cycloalkoxy group, —NR⁶R⁷, —CO₂R⁶, —CONR⁶R⁷, —SO₂R⁶, —SO₂NR⁶R⁷, —OCO₂R⁶, —OCONR⁶R⁷ and —NR⁶CO₂R⁷, and
R⁶ and R⁷ are the same or different and independently a hydrogen atom or a C₁₋₆ alkyl group optionally substituted with 1 to 2 carboxyl groups, wherein, when both R⁶ and R⁷ are optionally substituted C₁₋₆ alkyl groups, they may form a 3- to 12-membered cyclic amino group together with the nitrogen atom to which they are attached, and
R^{3C} is a hydrogen atom;
R¹ is a hydrogen atom;
R^{2A}, R^{2B}, R^{2C} and R^{2D} are all hydrogen atoms;
R⁸ is a methyl group,
or a pharmaceutically acceptable salt or solvate thereof.

62. The method according to claim 49, wherein the cancer stem cell inhibitor is napabucasin, or a pharmaceutically acceptable salt thereof.

63. The method according to claim 49, wherein the cancer stem cell inhibitor is 2,2′-((((((2-acetylnaphtho[2,3-b]furan-4,9-diyl)bis(oxy))bis(carbonyl))bis(azanediyl))bis(ethane-2,1-diyl))bis(azanediyl))diacetic acid, or a pharmaceutically acceptable salt thereof.

64. The method according to claim 49, wherein the immune checkpoint inhibitor is pembrolizumab.

65. The method according to claim 49, wherein
the cancer stem cell inhibitor is napabucasin or a prodrug thereof, or a pharmaceutically acceptable salt thereof,
the immune checkpoint inhibitor is pembrolizumab, and
the one or more patient characteristics comprise characteristics
(1) the cancer is right-sided colorectal cancer, and PD-L1 expression is positive on immune cells of the patient,
(2) the cancer is right-sided colorectal cancer, and PD-L1 expression is positive on tumor cells of the patient, or
(3) the cancer is right-sided colorectal cancer, and CMS of the patient is 1 or 4.

66. The method according to claim 65, wherein the cancer stem cell inhibitor is 2,2′-((((((2-acetylnaphtho[2,3-b]furan-4,9-diyl)bis(oxy))bis(carbonyl))bis(azanediyl))bis(ethane-2,1-diyl))bis(azanediyl)) diacetic acid, or a pharmaceutically acceptable salt thereof.
